(19) United States
(12) Patent Application Publication
(10) Pub. No.: US 2024/0199569 A1
(43) Pub. Date: Jun. 20, 2024

(54) PROCESS FOR THE MANUFACTURE OF INHIBITORS OF KRAS

(71) Applicant: Boehringer Ingelheim International GmbH, Ingelheim am Rhein (DE)

(72) Inventors: Yongda ZHANG, Sandy Hook, CT (US); Eugene CHONG, Ridgefield, CT (US); Jonathan Timothy REEVES, New Milford, CT (US); Jada A.H. WHITE, Southbury, CT (US); Zhen LEI, Ridgefield, CT (US)

(21) Appl. No.: 18/522,483

(22) Filed: Nov. 29, 2023

Related U.S. Application Data
(60) Provisional application No. 63/385,471, filed on Nov. 30, 2022.

(51) Int. Cl. C07D 317/72; C07D 413/04

(52) U.S. Cl. CPC C07D 317/72; C07D 413/04

(57) ABSTRACT

This invention relates to an efficient and enantioselective synthesis of the spiro-compounds of formula (1) as common intermediate, featuring an asymmetric introduction of the all-carbon quaternary centre in high selectivity and a very efficient one-pot procedure for the C2 elongation to generate the spiro-structure.

For the all-carbon quaternary centre, a Tsuji-Trost asymmetric allylic alkylation (AAA) process was designed to develop a sustainable procedure with very low catalyst loading, thereby also facilitating the Pd content control in the product, under practically solvent free conditions. Furthermore, multiple synthetic transformations in a one-pot procedure were implemented by using a one-pot procedure either in form of a three-transformation sequence comprising Hydroboration-Alkylation-Dieckmann Condensation to compound of formula (1) or in form of a five-transformation sequence comprising Hydroboration-Alkylation-Dieckmann Condensation-Hydrolysis-Decarboxylation to compound of formula (9).

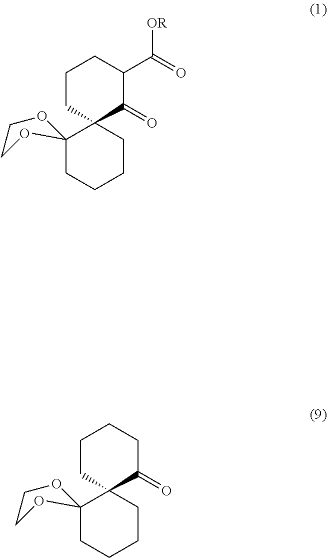

### TECHNICAL FIELD

The present invention relates to compounds and efficient and sustainable methods for preparing these compounds, which are useful as intermediates in the synthesis of KRAS inhibitors.

### BACKGROUND INFORMATION

A spiro-compound of formula (1), wherein R is defined as below,

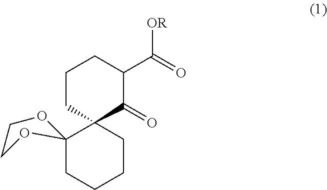

is for example an intermediate for a class of annulated 2-amino-3-cyano thiophenes and derivatives of formula (2) described in WO2023099624. Structurally, compound (1) contains a synthetically challenging spirocyclic ring system and an all-carbon quaternary chiral center, which are a challenge for the synthetic chemist (Jens Christoffers, Angelika Baro, Quaternary Stereocenters, Challenges and Solutions for Organic Synthesis, 1 ed. WILEY-VCH, 2005) especially when introduced in stereoselective manner for example by transition metal catalysed reactions.

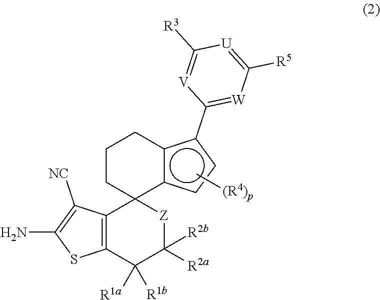

Compounds of formula (2) are used as inhibitors of KRAS in pharmaceutical compositions and preparations containing such compounds especially as agents for treatment and/or prevention of oncological diseases, e.g. cancer. This class of compounds has been found to possess anti-tumour activity, being useful in inhibiting the uncontrolled cellular proliferation which arises from malignant diseases. It is believed that this anti-tumor activity is, inter alia, derived from inhibition of KRAS mutated in position 12, preferably G12D and G12V mutant KRAS, or inhibition of KRAS wildtype amplified. Advantageously, the compounds can be selective for certain KRAS mutants, preferably KRAS G12D and G12V, or can be effective against a panel of KRAS mutants including KRAS wildtype amplified.

As this class of compounds is targeting an unmet medical need in the field of oncology, an efficient and scalable synthetic route to quickly supply kilogram quantities of the drug substance is of the utmost importance. Previously the route of synthesis reported was racemic relying on a chromatographic separation to obtain enantiopure compounds of formula (1), see Tetrahedron Letters, 2003, 44, 3333 or used a chiral auxiliary as in J. Chem. Soc. Perkin Trans 1, 1994, 3441.

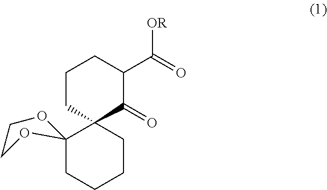

Thus, the aim was to find a process which provides the compound of formula (1) as central intermediate not only in enantioselective fashion but as an efficient and sustainable process using principals of green chemistry to provide easy access to this class of pharmaceutically desirable compounds of formula (2). Special focus was on one-pot procedures to on the one hand reduce the number of reagents and solvents used and on the other hand the number of isolation and purification steps. Additionally, also flow photochemistry set-up can be advantageous employed in a sustainable manufacturing process.

### BRIEF SUMMARY OF THE INVENTION

This invention relates to an efficient and enantioselective process for the spiro-compound of formula (1), wherein R is selected from the group consisting of linear, branched, or cyclic alkyl groups, and aryl groups as well as combinations thereof, preferably in all aforementioned definitions R has 1-12 carbon atoms or 1-6 carbon atoms, in particular R is a linear or branched alkyl C1-C12 or C1-C6 group optionally substituted, in addition in all aforementioned definitions R is preferably a saturated and unsubstituted hydrocarbon group; preferably R is methyl or ethyl, as central building block for compounds of formula (2) including novel earlier intermediates.

In another aspect, this invention relates to a short three step process of compound (1), wherein R is as defined above, featuring an asymmetric introduction of the all-carbon quaternary centre in high selectivity as step one, a carbonyl protection as step two and a very efficient one-pot procedure for the C2 elongation to generate the spiro-structure as step three. It has been found that the all-carbon quaternary centre can be prepared through a Tsuji-Trost asymmetric allylic alkylation (AAA) with unexpectedly low catalyst loading, thereby also facilitation the Pd content control in the product, under practically solvent free conditions. Furthermore, it has been surprisingly found that a one-pot procedure, meaning multiple synthetic transformations in one-reaction vessel without purification, in form of at least a three-transformation sequence of Hydroboration-Alkylation-Dieckmann Condensation can be used for a C2 elongation and ring closure to spiro-compound of formula (1), which is a central intermediate in the process to compounds of formula (2).

Furthermore, it was surprisingly found that it is possible to directly perform additional transformations without isolation of the product of the one-pot rection thereby converting the compound of formula (5) in a one-pot procedure comprising a five-transformation sequence of Hydroboration-Alkylation-Dieckmann Condensation-Saponification-Decarboxylation to the compound of formula (9).

Additionally, the C2 elongation can be achieved directly in a sustainable manner using a photoredox catalyzed radical hydroalkylation in a flow reaction set-up.

### DETAILED DESCRIPTION OF THE INVENTION

This invention relates to an efficient and enantioselective process of spiro-compounds of formula (1), wherein R is selected from the group consisting of linear, branched, or cyclic alkyl groups, and aryl groups as well as combinations thereof, preferably in all aforementioned definitions R has 1-12 carbon atoms or 1-6 carbon atoms, in particular R is a linear or branched alkyl C1-C12 or C1-C6 group optionally substituted, in addition in all aforementioned definitions R is preferably a saturated and unsubstituted hydrocarbon group; preferably R is methyl or ethyl, as central building block for compounds of formula (2).

In another aspect the invention relates to a short 3 step process to a compound of formula (1), as depicted in Scheme 1, starting from commercially available starting material. Special emphasis is not only on the very efficient and direct approach to a compound of formula (1) but also on the sustainability of the sequence by reduction of solvents and reagents needed during the reactions including a switch to environmentally benign solvents and reagents.

Furthermore, the use of additional solvent in the isolation and purification is avoided and recycling of used solvents significantly reduces the waste.

The first step introduces an all-carbon quaternary centre via a Tsuji-Trost asymmetric allylic alkylation (AAA) followed by carbonyl protection as second step and as third step a one-pot Hydroboration-Alkylation-Dieckmann Condensation (three-transformation) sequence.

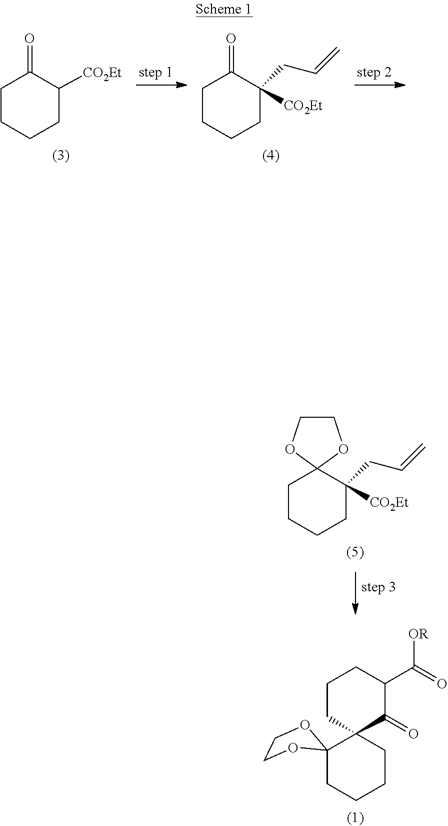

While the Tsuji-Trost asymmetric allylic alkylation has been described in literature (Chem. Rev. 2003, 103, 2921 and Org. Process Rec. Dev 2012, 16, 185) using a variety of Pd sources and chiral ligands to generate stereocenters including all-carbon quaternary stereo centers, no scalable process for the required transformation was reported in the literature (Adv. Synth. Catal. 2001, 343, 46). One aspect of this invention is a scalable process for the introduction of the quaternary stereo center using the principles of green chemistry. Trost reported allylation conditions (J. Am. Chem. Soc 1997, 119, 7879), which were used as starting point. In a ligand screening the (S,S)-DACH-Ph Ligand proves superior to the other tested ligands, compare table 1:

TABLE 1

| Entry | Ligand | (R)/(S) ratio |
|---|---|---|
| 1 | (S,S)-DACH-Ph Trost | >90:10 |
| 2 | (S,S)-DACH-Napthyl Trost | 78.4:21.6 |
| 3 | (S)-2-[2-[Bis(2-tolyl)phosphino]phenyl]-4-tert- | 60.7:39.3 |
| | butyl-4,5-dihydro-oxazole | |
| 4 | (1R,1′R,2S,2′S)-DuanPhos | 59.7:40.3 |
| 5 | (R,R)-DIPAMP | 60.6:39.4 |
| 6 | (R,R)-Me-DUPHOS | 58.5:41.5 |
| 7 | (S)-BINAP | 50.3:49.7 |
| 8 | (S,S)-NORPHOS | 53.8:46.2 |
| 9 | (R)-DIFLUORPHOS | 56.0:44.0 |
| 10 | (R)-SEGPHOS | 48.1:51.9 |

Furthermore, it was surprisingly found that the Pd loading can be reduced significantly up to 14 times less Pd can be used thus decreasing from 10,000 ppm to 700 ppm, while maintaining the good e.r. and conversion. Especially the sever reduction of the Pd load has a strong impact on the residual Pd control in the product, which is important as the heavy metal Pd needs to be strictly controlled in pharmaceutical product.

Furthermore, it was surprisingly found that practically solvent-free conditions (Chem. Rev. 2007, 107, 2503-2545) requiring only 4 equivalents of solvent leads to reduced reaction times in the process. As minimal amounts of solvent an organic solvent, or a mixture of an organic solvent and water can be used. Examples of organic solvents are known to the person skilled in the art and can be for example toluene or 2-Me-THF, preferably toluene. While only water as solvent leads to an incomplete conversion, it was surprisingly found that water is beneficial for a full conversion in organic solvent. Preferably a 100:1 to 400:1 mol ratio of water:Pd, is used in the reaction, more preferably a 160:1 to 200:1 mol ratio of water:Pd. Due to the higher concentration the reaction time is reduced, and it is possible to lower the Pd loading even below 0.250 mol %, preferably below 0.125 mol %, more preferably below 0.05 mol % and most preferably to approximately 0.035 mol %.

It was surprisingly found that a temperature around 10° C. leads to better conversion while maintaining the good e.r., see table 2:

TABLE 2

| Temperature [° C.] | Conversion after 120 min [%] |
|---|---|
| 0 | 86 |
| 10 | 93 |
| 21 | 88 |
| 30 | 82 |

It was found that the base can be chosen from commonly known nitrogen bases like TMG, DBU, triethylamine, Hünig's base, pyridine, piperidine, morpholine, DABCO, etc., preferably TMG is used.

Preferably TMG is used in an amount between 1.0 to 3.0 equivalents, more preferably between 1.5 to 2.5 equivalents, most preferably approximately 2.0 eq.

The ally reagent can be chosen from the non-exhaustive list of allyl acetates, ally α-halogen acetates like allyl 2-chloroacetate, allyl carbonates like ally methyl carbonate, ally tert-butylcarbonate, allyl (2,2,2-trichloroethyl) carbonate, ally benzyl carbonate, diallyl carbonate. Preferably ally acetate is used for the reaction.

Furthermore, additional organic solvent for extraction is eliminated in the isolation; in comparison, unsafe diethyl ether was used in the original Trost procedure. The resulting outcome greatly minimizes solvent waste to the environment from the original Trost procedure.

In another aspect it was found that the second step, the ketal protection, can be performed at ambient temperature with ethylene glycol as the reactant and solvent, thus avoiding the classic time-consuming azeotropic distillation at high temperature in toluene and thus reacting under ambient conditions a compound of formula (4) to give a compound of formula (5).

In another aspect it was found that the third step can convert a compound of formula (5) directly to a compound of formula (1) by a one-pot Hydroboration-Alkylation-Dieckmann Condensation (three-transformation) sequence, which very efficiently introduces a C2 elongation, followed by ring closure to form the spiro-compound of formula (1), as shown in Scheme 2 below:

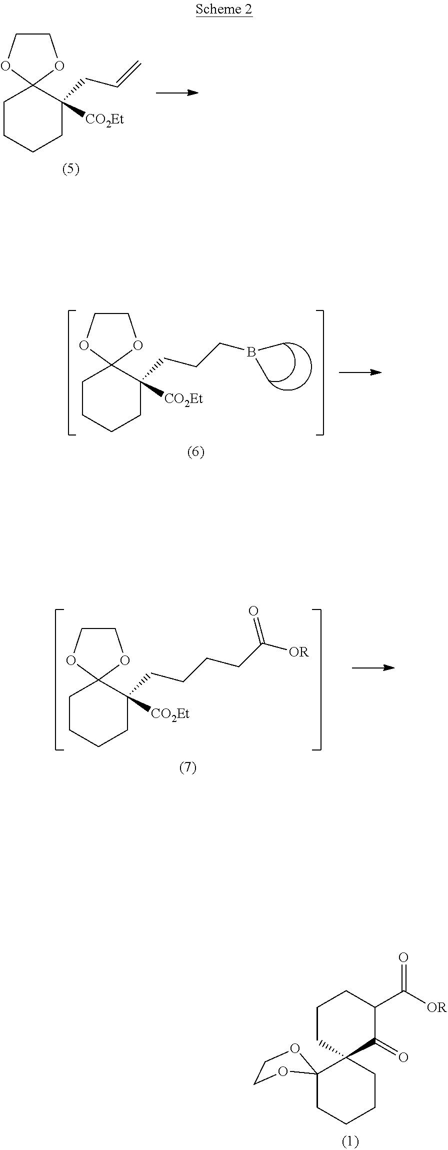

Brown reported in J. Am. Chem. Soc 1969, 91, 2146 more than 50 years ago an ester synthesis relying on Hydroboration followed by Alkylation. However, yields were moderate due to competition of the different B-alkyl bond migrations and for complete conversion the di-halogen acetates were used thereby producing the corresponding α-halogenated esters as products.

It was found in this invention that this can be overcome by careful selection of the reaction sequence and temperature profile.

As sterically hindered boranes such as 9-BBN proved superior, 9-BBN was exclusively used for the hydroboration of the first transformation in the one-pot sequence.

It was found that as alkylating reagent α-halo acetate can be used where halogen is selected from Cl, Br or I, preferably Cl. The acetate can be different alkyl or aryl esters as described for R above, preferably benzyl, ethyl or methyl α-halo acetate.

Furthermore, it was found that at least one base is needed for the alkylation as well as the Dieckmann Condensation reaction. The base for each transformation can be selected from KOtBu, NaOtBu, KaOH, NaOH, LiOH, LiOtAmyl, NaOtAmyl, NHMDS, KHMDS, LHMDS or LDA.

Additionally, it was surprisingly found that in the one-pot procedure only the use of NHMDS, KHMDS, or LHMDS for alkylation and Dieckmann Condensation gave satisfying results. When LHMDS is used in super stochiometric amounts in the alkylation of the borane it can directly induce the Dieckmann Condensation to form the spiro compound by closing the second 6-membered ring. The amount of base used in total is between 3.0 to 6.0 equivalents, preferably between 3.1 to 4.0 equivalents, more preferably approximately 3.3 equivalents.

In another aspect of this invention, it was found that at this stage of the one-pot procedure the product of the Dieckmann Condensation, the compound of formula (1), can either be isolated as described above or additional two transformations can be performed without isolation or purification to react the compound of formula (1) to a compound of formula (9), as shown in Scheme 3 below:

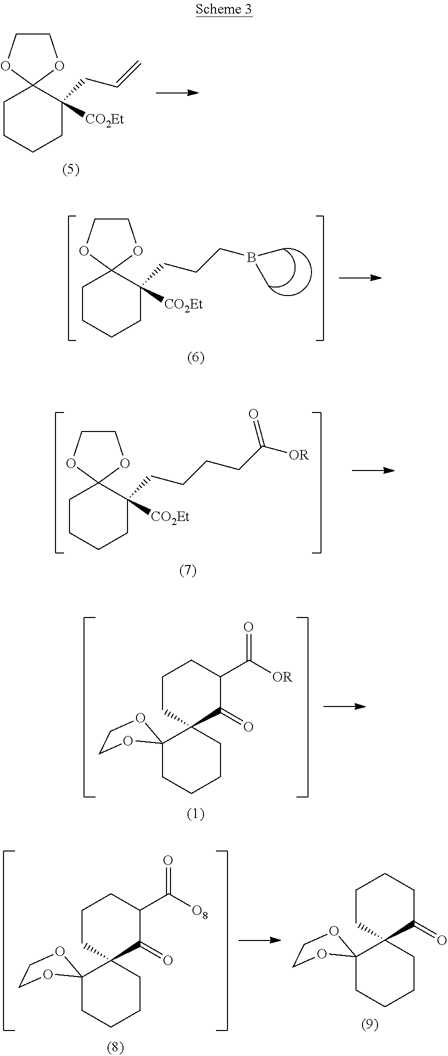

It was found that direct addition of aqueous base to (1) and increase in temperature leads to saponification of the ester moiety of the compound of formula (1) followed by decarboxylation giving a compound of formula (9). Thus, preforming five transformations in one pot.

In another aspect it was found that the double bond of compound of formula (5) can react in a radical hydroalkylation as described by JACS, 2021, 143, 11251 using dual HAT (hydrogen atom transfer) catalysis with an electron deficient/acidic C—H. This results in a C2 elongation of the unactivated double bond.

As electron deficient C—H for example 1,3-dicarbonyl compounds, ß-keto esters, β-keto amide, β-keto nitrile, cyanoacetate, and malonic acid diesters can be used, preferable malonic acid dialkylesters and Meldrum's acid (25) are used.

As solvents trifluoro toluene, dimethylacetamide, dimethyl sulfoxide, N-methyl-2-pyrrolidone, and acetonitrile can be employed, preferably acetonitrile.

Commonly used photoredox catalyst are for example Ir or Ru complexes, which rely on the redox potential of the metal center for their reactivity. However heavy metal contamination is tightly regulated in pharmaceutical products due to toxicological issues. Therefore, metal free photocatalysts were tested to find a sustainable option for toxicological, environmental and economic reasons. Metal free photocatalysts can be picked from the non-exhaustive list of 5CzBN (penta-carbazolylbenzonitrile), 4CzIPN (2,4,5,6-Tetrakis(9H-carbazol-9-yl) isophthalonitrile) 3CzCIIPN (2,4,6-Tri(9H-carbazol-9-yl)-5-chloroisophthalonitrile), and 3DPAFIPN (2,4,6-Tris(diphenylamino)-5-fluoroisophthalonitrile). Most preferred is the use of 4CzIPN (26).

As H-atom donor a thiol reagent in its disulfide form is described however it was reasoned that switching to a monomeric species might increase the reaction speed. Therefore, thiol reagents that are not prone to form disulfides were tested like sterically hindered aromatic thiols, trialkyl silanethiols and triaryl silanethiols, most preferred is triphenylsilanethiol (27). Different borane catalysts of 3-quinuclidinol-borane type can be utilized to generate the nucleophilic borane radical of the catalytic cycle, most preferred is 3-quinuclidinol-borane (28).

In a further aspect the photochemical reaction can be run in a flow chemistry process. This has many advantages over a batch process as previously also described in Chem. Rev. 2016, 116, 17, 10276-10341 like a large surface area for the photochemical reaction and easier temperature control. By using a flow reactor, the reaction scale can be increased to kg scale for the photo reaction. Employing a continues flow process as manufacturing process avoids multiple cleaning procedures, waiting and hold times and is preferred from an economic and environmental perspective.

The compound of formula (29) is isolated as direct product of the photochemical hydroalkylation and can be further transformed without additional purification via decarboxylative ring opening into the acid of formula (31) and its corresponding ester (7), which is also an intermediate in the one-pot reactions described above. The decarboxylative ring opening can be achieved in the crude product solution by addition of CDI and elevating the temperature. The free acid moiety of the compound of formula (31) can be subjected to esterification to generate the compound of formula (7). Suitable reaction conditions for this transformation are known in the art.

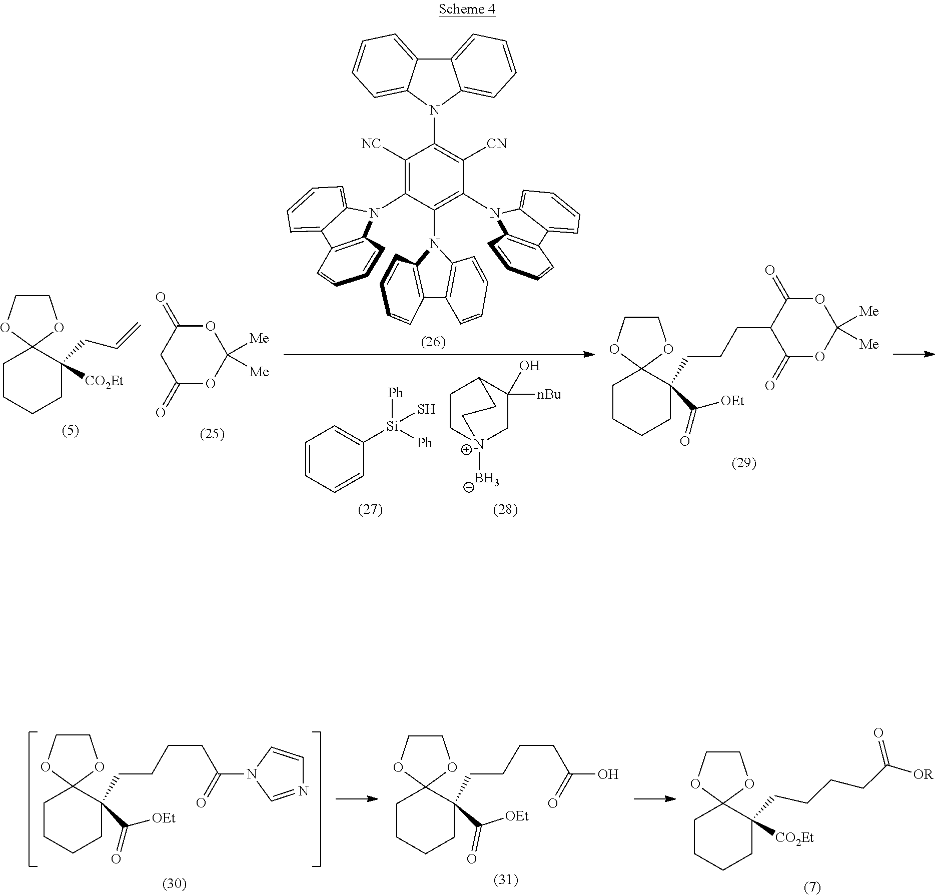

The compound of formula (7) can be transformed under the conditions described above into either compound (1) directly or in the compound of formula (9) by a one-pot reaction for 3 transformations. It was surprisingly found that no further adjustments of the reaction conditions from (7) to (9) are necessary. This highlights the robustness of the transformations either in one pot from isolated (7) as in Scheme 5 or as described previously in the one-pot five transformation sequence in scheme 3.

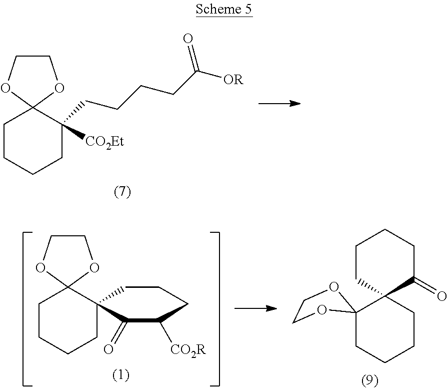

The compound of formula (9) can optionally be crystallized at low temperatures from heptane to generate a crystalline product in excellent optical purity or directly used for further transformations.

In another aspect, this invention relates to an efficient and enantio- and regioselective process of the spiro-compounds of formula (10) or (11) including novel intermediates.

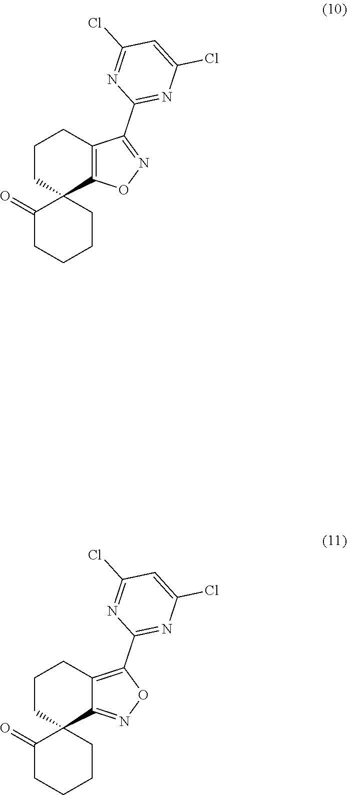 [SMILES: O=C1CCCC[C@@]12CCCc1c(-c3nc(Cl)cc(Cl)n3)noc12 ; O=C1CCCC[C@@]12CCCc1c2noc1-c1nc(Cl)cc(Cl)n1]

It was found that starting either from a compound of formula (1) or a compound of formula (9) the different corresponding isoxazole regioisomers can be generated. These can be further converted to spiro-compounds of formula (10) or (11), which are on the one hand densely functionalized compounds that can be selectively and efficiently converted into any desirable compound of formula (2) in a highly convergent manner and are on the other hand readily available in the synthetic process described above.

In one aspect of the invention, as shown in Scheme 4 below, starting from a compound of formula (9) first C2 elongation with ethyl oxalate gives a compound of formula (12) which can react with hydroxylamine without isolation to a compound of formula (13) by heteroaryl ring formation to give the isoxazole ring. The ester moiety of a compound of formula (13) can further be converted by treatment with ammonium hydroxide to the carboxamide moiety of a compound of formula (14). Dehydration of a compound of formula (14) leads to a compound of formula (15) featuring a nitrile. The pyrimidine ring of the compound of formula (17) can then be formed by reaction with nitrogen source and conversion of the not isolated amidine intermediate of formula (16) with malonate. The Hydroxy groups of a compound of formula (17) can be converted to the di-chloride of formula (10). Such transformations are known to one skilled in the art and include, among others, those described herein.

In another aspect, this invention relates to the use of compound of formula (10) as intermediates in the synthesis for KRAS inhibitors, preferably annulated 2-amino-3-cyano thiophenes and derivatives of formula (2).

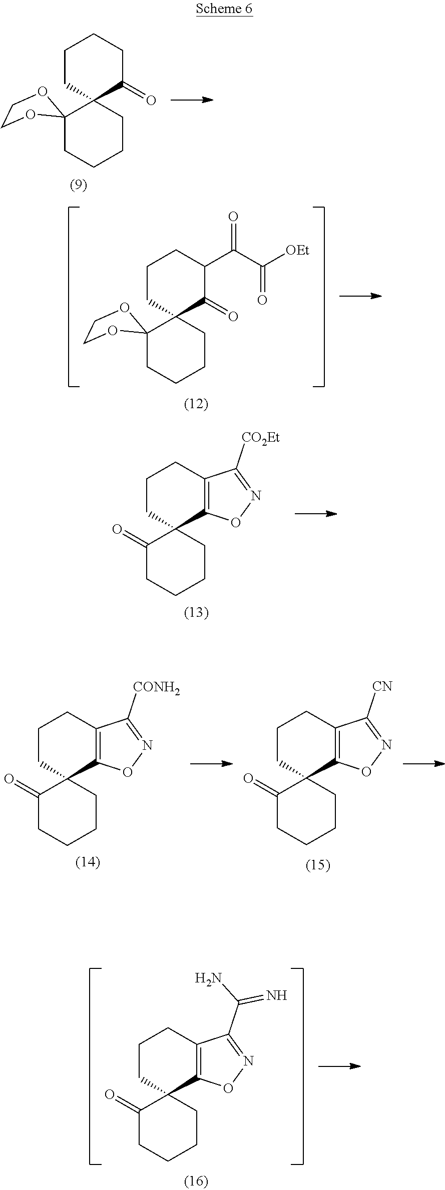

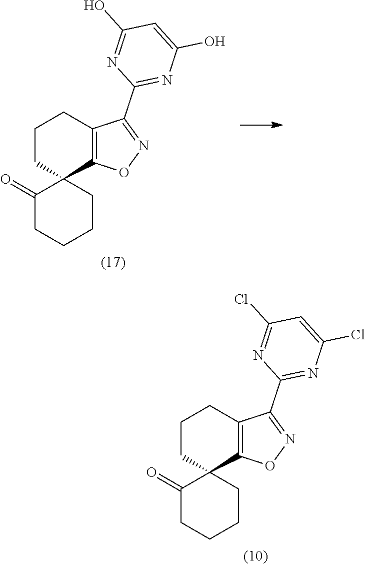 [SMILES: O=C1CCCC[C@@]12CCCc1c(-c3nc(O)cc(O)n3)noc12 ; O=C1CCCC[C@@]12CCCc1c(-c3nc(Cl)cc(Cl)n3)noc12]

In another aspect of the invention as shown in Scheme 5 below, starting from a compound of formula (1) reaction with hydroxylamine generates the isoxazole heteroaryl ring in a compound of formula (18a). Next the triflate is introduced by reaction of compound of formula (18a) to a compound of formula (19). Carboxylation of the triflate of formula (19) leads to a compound of formula (20) comprising an ester moiety. The ester moiety can be further converted via the carboxamide of formula (20) to a nitrile of formula (22). The pyrimidine ring of the compound of formula (24) can then be formed by reaction with nitrogen source and conversion of the not isolated amidine intermediate of formula (23) with malonate. The Hydroxy groups of a compound of formula (24) can be converted to the di-chloride of formula (11). Such transformations are known to one skilled in the art and include, among others, those described herein.

In another aspect, this invention relates to the use of compound of formula (11) as intermediates in the synthesis for KRAS inhibitors, preferably annulated 2-amino-3-cyano thiophenes and derivatives of formula (2).

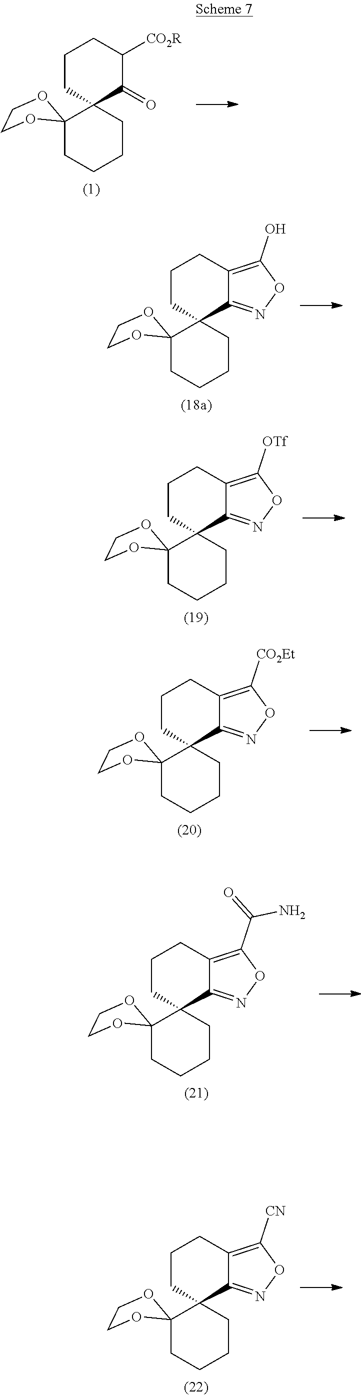

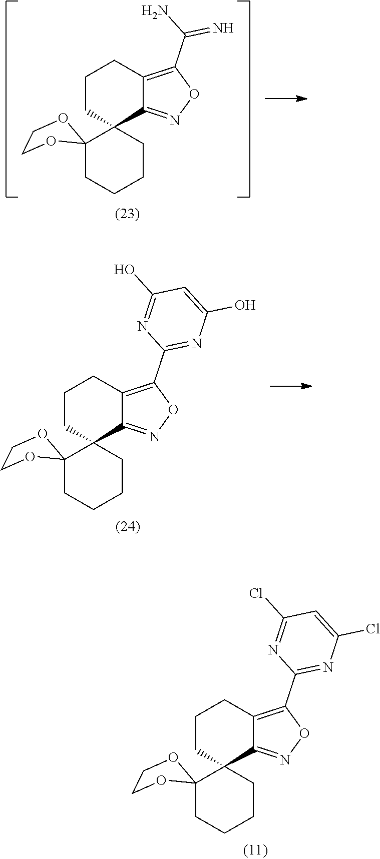

### PREFERRED EMBODIMENTS

In one embodiment, this invention relates to a compound of formula (1),

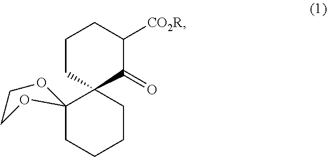

wherein R is selected from the group consisting of linear, branched, or cyclic alkyl groups, and aryl groups as well as combinations thereof, preferably in all aforementioned definitions R has 1-12 carbon atoms or 1-6 carbon atoms, in particular R is a linear or branched alkyl C1-C12 or C1-C6 group optionally substituted, in addition in all aforementioned definitions R is preferably a saturated and unsubstituted hydrocarbon group; preferably R is methyl or ethyl.

In another embodiment, this invention relates to the process for the preparation of a compound of formula (1)

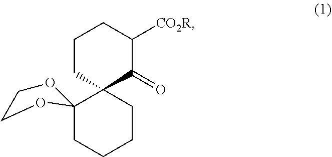

wherein R is as defined in the previous embodiment, and the process comprises reacting a compound of formula (5) in a one-pot reaction.

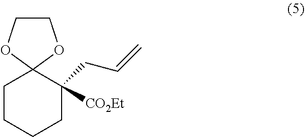 [SMILES: C=CC[C@]1(C(=O)OCC)CCCCC12OCCO2]

In another embodiment, this invention relates to the process according to the previous embodiment, where a one-pot sequence from a compound of formula (5) to a compound of formula (1) comprises at least 3 transformations: Hydroboration, Alkylation, and Dieckmann Condensation.

In a further embodiment, this invention relates to the process according to the previous embodiments, wherein the hydroboration is carried out in the presence of 9-BBN.

In a preferred embodiment, this invention relates to the process according to the previous embodiments, where the 9-BBN is used in 1.2 equivalents.

In a further embodiment, this invention relates to the process according to the previous embodiments, wherein the alkylation is carried out in the presence of an α-halo acetate, preferably ethyl or methyl α-halo acetate.

In a preferred embodiment the α-halo acetate used is selected from Cl, Br or I, preferably Cl. In a further embodiment, this invention relates to the process according to the previous embodiments, wherein each of the transformations of alkylation and condensation is carried out in the presence of at least one base, optionally a different base for the different transformations.

In a preferred embodiment, this invention relates to the process according to the previous embodiments, where the base used for the hydroboration-alkylation-Dieckmann Condensation sequence is selected from NHMDS, KHMDS or LHMDS, preferably LHMDS.

In a further embodiment, this invention relates to the process according to the previous embodiments, where the amount of base used in total is between 3.0 to 6.0 equivalents, preferably between 3.1 to 4.0 equivalents, more preferably approximately 3.3 equivalents.

In another embodiment, this invention relates to the process for the preparation of a compound of formula (1)

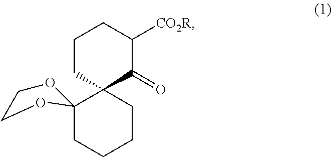

wherein R is as defined in the previous embodiment, and the process comprises reacting a compound of formula (5) in a photochemical reaction.

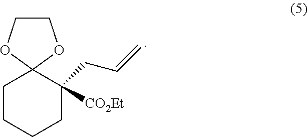 [SMILES: C=CC[C@]1(C(=O)OCC)CCCCC12OCCO2]

In a further embodiment, this invention relates to the process according to the previous embodiment, wherein a flow process is used for the photochemical reaction.

In a further embodiment, this invention relates to the process according to the previous embodiments, wherein the photochemical reaction uses 3-dicarbonyl compounds, ß-keto esters, β-keto amide, β-keto nitrile, cyanoacetate, and malonic acid diesters, preferably malonic acid dialkylesters or Meldrum's acid are used.

In a further embodiment, this invention relates to the process according to the previous embodiments, wherein the photochemical reaction uses trifluoro toluene, dimethylacetamide, dimethyl sulfoxide, N-methyl-2-pyrrolidone, and acetonitrile as solvent, preferably acetonitrile.

In a further embodiment, this invention relates to the process according to the previous embodiments, wherein the photochemical reaction uses 5CzBN (penta-carbazolylbenzonitrile), 4CzIPN (2,4,5,6-Tetrakis(9H-carbazol-9-yl) isophthalonitrile) 3CzCIIPN (2,4,6-Tri(9Hcarbazol-9-yl)-5-chloroisophthalonitrile), or 3DPAFIPN (2,4,6-Tris(diphenylamino)-5-fluoroisophthalonitrile) as photochemical catalyst, preferably 4CzIPN (26).

In a further embodiment, this invention relates to the process according to the previous embodiments, wherein the photochemical reaction uses trialkyl or triaryl silanethiols, preferably triphenylsilanethiol.

In a further embodiment, the photochemical reaction is carried out in the presence of a 3-quinuclidinol-borane, preferably 3-quinuclidinol-borane (28)

In a further embodiment, this invention relates to the process according to the previous embodiments, wherein the compound of formula (5) is prepared from a compound of formula (3).

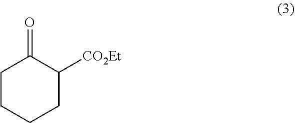 [SMILES: CCOC(=O)C1CCCCC1=O]

Preferably the compound of formula (5) is prepared by asymmetric allylic alkylation of a compound of formula (3).

In a further embodiment, this invention relates to the process according to the previous embodiments, where the asymmetric allylic alkylation of a compound of formula (3) uses Allylpalladium(II) chloride dimer below 0.250 mol %, preferably below 0.125 mol %, more preferably below 0.050 mol % and most preferably approximately 0.035 mol %.

In a preferred embodiment, this invention relates to the process according to the previous embodiments, where the asymmetric allylic alkylation of a compound of formula (3) uses (S,S)-DACH-Ph Trost ligand in a Pd/Ligand ratio of between 1.00:1.00 to 1.00:3.00, preferably between 1.00:1.07 to 1.00:1.20, most preferably of approximately 1.00:1.15.

In a further embodiment, this invention relates to the process according to the previous embodiments, where the asymmetric allylic alkylation of a compound of formula (3) uses toluene or MeTHF as solvent, preferably toluene.

In a preferred embodiment, this invention relates to the process according to the previous embodiments, where the asymmetric allylic alkylation of a compound of formula (3) uses toluene as solvent, preferably 4 equivalents of toluene.

In a further embodiment, this invention relates to the process according to the previous embodiments, where the asymmetric allylic alkylation of a compound of formula (3) uses between 1.0 to 3.0 equivalents of TMG, preferably between 1.5 to 2.5 equivalents, most preferably approximately 2.0 eq of TMG.

In a further embodiment, this invention relates to the process according to the previous embodiments, where the asymmetric allylic alkylation of a compound of formula (3) is carried out between 5 to 20° C., most preferably between 10 to 15° C. reaction temperature.

In a further embodiment, this invention relates to the process according to the previous embodiments, where the asymmetric allylic alkylation of a compound of formula (3) is carried out either under water free conditions or in an organic solvent system with a mol ratio between 100:1 to 400:1 of water:Pd, preferably a mol ratio between 160 to 200:1 of water:Pd.

According to another embodiment, this invention relates to the process according to the previous embodiments, wherein the one-pot sequence of a compound of formula (5) has a compound of formula (9) as isolated product and comprises 5 transformations: Hydroboration, alkylation, Dieckmann Condensation, saponification, decaboxylation.

In another embodiment, this invention relates to the process for the preparation of a compound of formula (11)

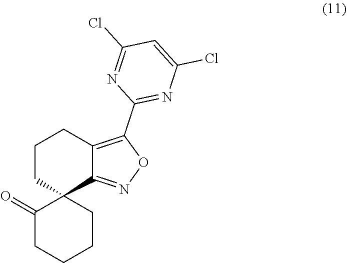 [SMILES: O=C1CCCC[C@@]12CCCc1c2noc1-c1nc(Cl)cc(Cl)n1]

comprising the compound of formula (1), wherein R is as defined as above, as intermediate.

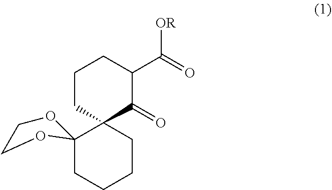

The compound of formula (1) is generated according to any one of the previous embodiments of the process of the invention.

In a further embodiment, this invention relates to the process according to the previous embodiments, wherein a compound of formula (11) is synthesized from a compound of formula (24).

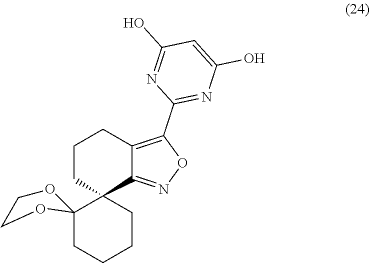 [SMILES: Oc1cc(O)nc(-c2onc3c2CCC[C@@]32CCCCC23OCCO3)n1]

In a further embodiment, this invention relates to the process according to the previous embodiments, wherein a compound of formula (24) is synthesized from a compound of formula (22). 5

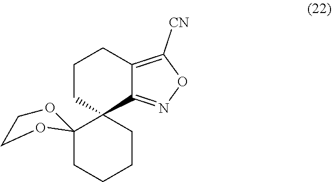 [SMILES: N#Cc1onc2c1CCC[C@@]21CCCCC12OCCO2]

In a further embodiment, this invention relates to the process according to the previous embodiments, wherein a compound of formula (22) is synthesized from a compound of formula (21).

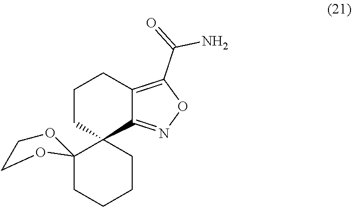 [SMILES: NC(=O)c1onc2c1CCC[C@@]21CCCCC12OCCO2]

In a further embodiment, this invention relates to the process according to the previous embodiments, wherein a compound of formula (21) is synthesized from a compound of formula (20).

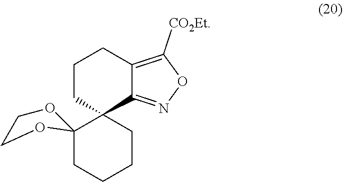 [SMILES: CCOC(=O)c1onc2c1CCC[C@@]21CCCCC12OCCO2]

In a further embodiment, this invention relates to the process according to the previous embodiments, wherein a compound of formula (20) is synthesized from a compound of formula (19).

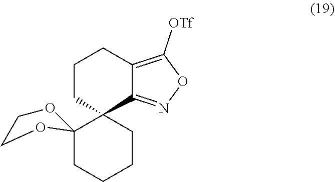 [SMILES: O=S(=O)(Oc1onc2c1CCC[C@@]21CCCCC12OCCO2)C(F)(F)F]

In a further embodiment, this invention relates to the process according to the previous embodiments, wherein a compound of formula (19) is synthesized from a compound of formula (18a).

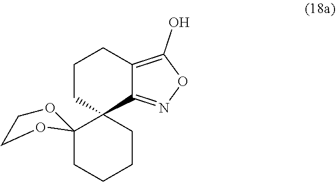 [SMILES: Oc1onc2c1CCC[C@@]21CCCCC12OCCO2]

In a further embodiment, this invention relates to the process according to the previous embodiments, wherein a compound of formula (18a) is present as its tautomeric form of compound of formula (18b).

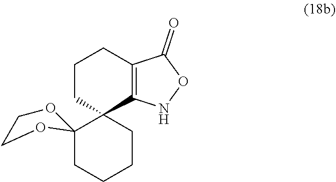 [SMILES: O=c1o[nH]c2c1CCC[C@@]21CCCCC12OCCO2]

In another embodiment, this invention relates to the process for the preparation of a compound of formula (10)

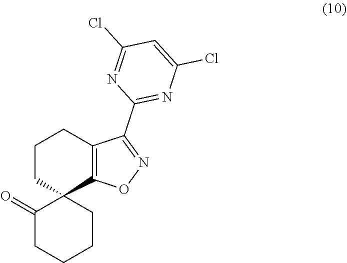 [SMILES: O=C1CCCC[C@@]12CCCc1c(-c3nc(Cl)cc(Cl)n3)noc12]

comprising the compound of formula (9) as intermediate.

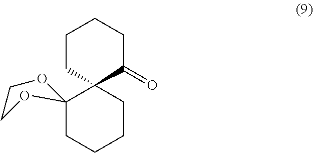 [SMILES: O=C1CCCC[C@@]12CCCCC21OCCO1]

The compound of formula (9) is generated according to one of the previous embodiments. In a further embodiment, this invention relates to the process according to the previous embodiments, wherein a compound of formula (10) is synthesized from a compound of formula (17).

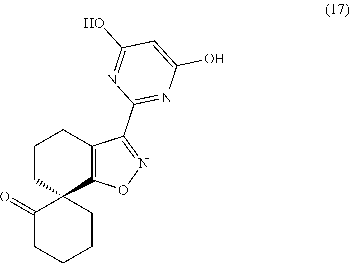 [SMILES: O=C1CCCC[C@@]12CCCc1c(-c3nc(O)cc(O)n3)noc12]

In a further embodiment, this invention relates to the process according to the previous embodiments, wherein a compound of formula (17) is synthesized from a compound of formula (15).

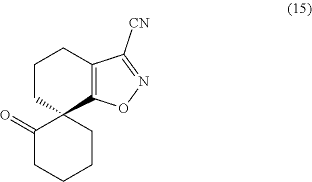 [SMILES: N#Cc1noc2c1CCC[C@@]21CCCCC1=O]

In a further embodiment, this invention relates to the process according to the previous embodiments, wherein a compound of formula (15) is synthesized from a compound of formula (14).

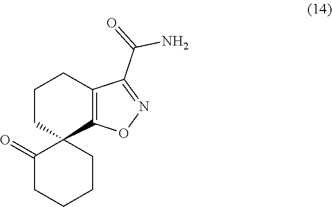 [SMILES: NC(=O)c1noc2c1CCC[C@@]21CCCCC1=O]

In a further embodiment, this invention relates to the process according to the previous embodiments, wherein a compound of formula (14) is synthesized from a compound of formula (13).

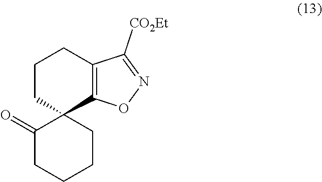 [SMILES: CCOC(=O)c1noc2c1CCC[C@@]21CCCCC1=O]

In a further embodiment, this invention relates to the process according to the previous embodiments, wherein a compound of formula (13) is synthesized from a compound of formula (12).

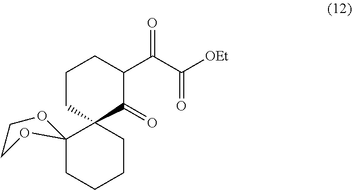

In another embodiment, this invention relates to one of the compounds selected from compound of formula (10), compound of formula (17), compound of formula (15), compound of formula (14), compound of formula (13), and compound of formula (12).

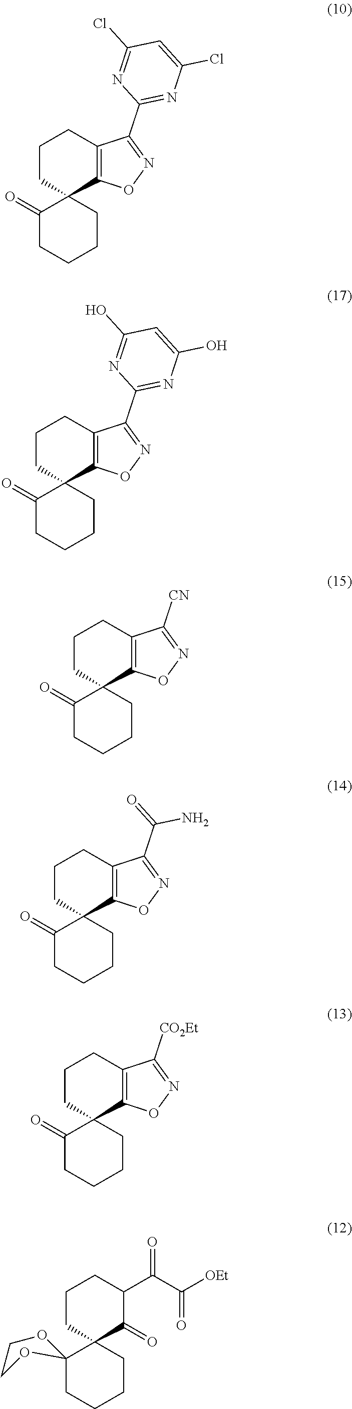

In another embodiment, this invention relates to one of the compounds selected from compound of formula (11), compound of formula (24), compound of formula (22), compound of formula (21), compound of formula (20), compound of formula (19), compound of formula (18a), and compound of formula (18b).

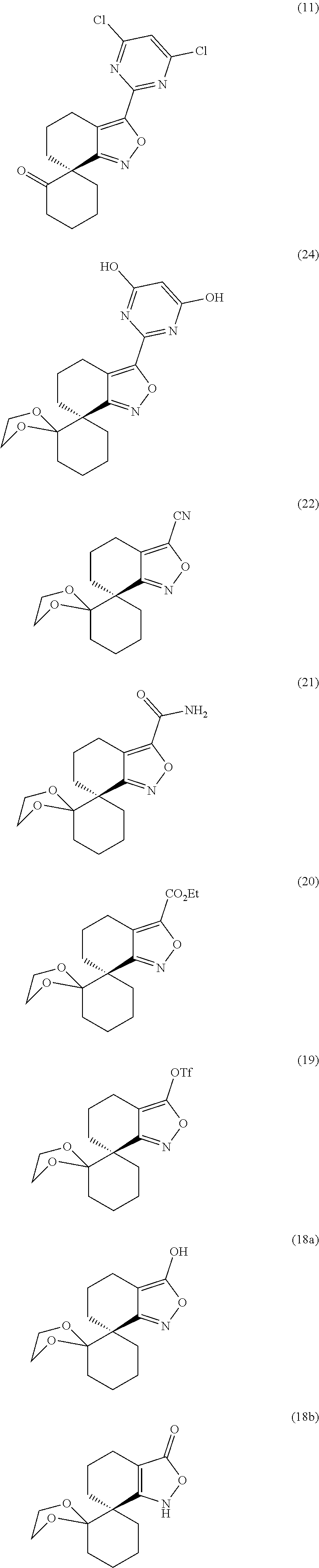

In another embodiment, this invention relates to the crystalline compound of formula (9), the crystalline compound of formula (15), the crystalline compound of formula (14), and the crystalline compound of formula (13).

In another embodiment, this invention relates to the use of any of the compounds of the previous embodiments as intermediates in the synthesis for KRAS inhibitors, preferably annulated 2-amino-3-cyano thiophenes and derivatives of formula (2).

### Used Terms and Definitions

The term “alkyl” stands for a hydrocarbon moiety and includes acyclic, saturated, branched or linear hydrocarbon moieties, which can optionally be further substituted.

The term “aryl” as used herein, denotes a carbocyclic aromatic monocyclic group containing 6 carbon atoms which is optionally further substituted.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like. One or more substituents can be selected from the group consisting of fluorine, chlorine, bromine, NC—, F₃C—, C₁₋₃-alkyl-, CH₃—O—C₁₋₃-alkylene-, C₁₋₃-alkyl-O— and phenyl.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis.

The term “one-pot procedure” or “one pot-reaction”, means herein that multiple synthetic steps/transformations are preformed sequentially in one-reaction vessel without purification, by subjecting the reactant to successive chemical reaction conditions in just one reactor. The term mol ratio of water:Pd used within refers to the mol ratio of mol water to mol palladium taking into account that the allylpalladium(II) chloride dimer contains two mol of Pd. The term Pd/Ligand ratio stands for the mol ratio in the active catalytic species formed in the reaction from allylpalladium(II) chloride dimer and the (S,S)-DACH-Ph Trost ligand taking into account that the dimer contains two mol of Pd.

Abbreviation used herein:

| | |
|---|---|
| Ac | acetyl |
| ACN | acetonitrile |
| aq. | aquatic, aqueous |
| 9-BBN | 9-Borabicyclo[3.3.1]nonane |
| Bn | benzyl |
| Boc | tert-butyloxycarbonyl |
| Bu | butyl |
| c | concentration |
| CDI | Carbonyldiimidazole |
| d | day(s) |
| TLC | thin layer chromatography |
| DABCO | 1,4-diazabicyclo(2.2.2)octane |
| DCM | dichloromethane |
| DIPEA | N-ethyl-N,N-diisopropylamine (Hünig's base) |
| DMAP | 4-N,N-dimethylaminopyridine |
| DME | 1,2-dimethoxyethane |
| DMF | N,N-dimethylformamide |
| DMSO | Dimethyl sulfoxide |
| dppf | 1.1′-bis(diphenylphosphino)ferrocene |
| equiv. | equivalent(s) |
| er/e.r. | Enantiomeric ratio |
| ESI | electron spray ionization |
| Et | ethyl |
| EtOAc | ethyl acetate |
| EtOH | ethanol |
| h | hour(s) |
| HPLC | high performance liquid chromatography |
| IPA | Iso-propanol |
| conc. | concentrated |
| KHMDS | potassium bis(trimethylsilyl)amide |
| LC | liquid chromatography |
| LDA | lithium diisopropylamide |
| LHMDS/ | lithium bis(trimethylsilyl)amide |
| LiHMDS | |
| Me | methyl |
| MeCN | Acetonitrile |
| MeOH | methanol |
| MeTHF | 2-methyltetrahydrofuran |
| min | minute(s) |
| MS | mass spectrometry |
| MTBE | Methyl tert-butyl ether |
| NHMDS | sodium bis(trimethylsilyl)amide |
| NMR | nuclear magnetic resonance |
| NP | normal phase |
| n.a. | not available |
| Ph | phenyl |
| Py | pyridine |
| rac | racemic |
| Rf (R_{f}) | retention factor |
| RP | reversed phase |
| rt | ambient temperature |
| s | second(s) |
| tBu | tert-butyl |
| TEA | triethyl amine |
| temp. | temperature |
| t/tert | tertiary |
| Tf | triflate |
| TFA | trifluoroacetic acid |
| TFAA | trifluoroacetic anhydride |
| THF | tetrahydrofuran |
| TMG | 1,1,3,3-Tetramethylguanidine |
| TMSCl | Trimethylsilyl chloride |
| t_{Ret.} | retention time (HPLC) |
| Ts | tosylate |
| UPLC | ultra performance liquid chromatography |
| UV | ultraviolet |
| wt | weight |

### EXAMPLES

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon). If a compound is to be represented both by a structural formula and by its nomenclature, in the event of a conflict the structural formula is decisive.

### Chromatography

The thin layer chromatography is carried out on ready-made silica gel 60 TLC plates on glass (with fluorescence indicator F-254) made by Merck.

The analytical HPLC (reaction control) of intermediate and final compounds is carried out using columns made by Waters (names: XBridge™ C18, 2.5 μm, 2.1×20 mm or XBridge™ C18, 2.5 μm, 2.1×30 mm or Aquity UPLC BEH C18, 1.7 μm, 2.1×50 mm) and YMC (names: Triart C18, 3.0 μm, 2.0×30 mm) and Phenomenex (names: Luna C18, 5.0 μm, 2.0×30 mm).

HPLC Method I

| HPLC | Agilent 1100/1200 system | |
|---|---|---|
| Detection | 210 nm (bandwidth 10 nm, reference 500 nm) | |
| signal | | |
| Column | Waters, Xbridge BEH C18, 2.5 μm, 4.6 × 100 mm column | |
| Column | 30° C. | |
| temperature | | |
| Solvent | A: 0.1% (v/v) HClO₄ in HPLC grade water | |
| | B: ACN HPLC grade | |
| Flow | 1.2 mL/min | |
| Gradient | 0 min | 20% B |
| | 10 min | 65% B |
| | 12 min | 80% B |
| | 14 min | 90% B |
| | 15 min | 90% B |

HPLC Method II

| HPLC | Agilent 1100/1200 system | |
|---|---|---|
| Detection | 210 nm (bandwidth 10 nm, reference 500 nm) | |
| signal | | |
| Column | Waters, Xbridge BEH C18, 2.5 μm, 4.6 × 100 mm column | |
| Column | 30° C. | |
| temperature | | |
| Solvent | A: 0.1% (v/v) HClO₄ in HPLC grade water | |
| | B: ACN HPLC grade | |
| Flow | 1.2 mL/min | |
| Gradient | 0 min | 25% B |
| | 10 min | 55% B |
| | 12 min | 80% B |
| | 14 min | 90% B |
| | 15 min | 90% B |

Chiral Method I:

| | |
|---|---|
| GC | HP 7890 GC |
| Column | CHIRALDEX, G-TA, Cat. No. 73033AST, Col: 262953-01 |

(Length: 30 m, Internal Diameter: 0.25 mm, Film thickness: 0.12 μm)

| | |
|---|---|
| Detector | FID |
| Inlet | Split ratio = 50:1 |
| Temperature | 140° C. |
| Carrier Gas | Helium, constant flow, 1.5 ml/min |

Oven Temperature: Initial 95° C. hold for 5 min, and ramp to 160° C.
at a rate of 10° C./min and hold for 10 min.

| | |
|---|---|
| Run time | 21.5 min |
| FID Temp | 300° C. |
| H2 flow | 35 mL/min; |
| Air flow | 400 mL/min |
| Makeup flow | on 20 mL/min |
| Mode | Constant makeup and fuel flow |

Chiral Method II:

| SFC | Agilent 1260 SFC system | |
|---|---|---|
| Detection | 210 nm (bandwidth 10 nm, reference 500 nm) | |
| signal | | |
| Column | Lux Cellulose-2, Phenomenex (4.6 × 100 mm, 5 micron | |
| | particle size) | |
| Column | 35° C. | |
| temperature | | |
| Mobile phase | A: CO₂, bone-dry, without dip-tube (Air Gas UN1013) | |
| | B: IPA | |
| Flow | 2 mL/min | |
| Gradient | 0 min | 1% B |
| | 3 min | 3% B |
| | 8 min | 50% B |
| | 9.5-10 min | 1% B |
| | 10 min | 1% B |

| | |
|---|---|
| Pressure limit | 20-550 bar |

Chiral Method III:

| SFC | Agilent 1260 SFC system | | |
|---|---|---|---|
| Detection | 210 nm (bandwidth 10 nm, reference 500 nm) | | |
| signal | | | |
| Column | ChiralPak IF (4.6 × 100 mm, 5 micron particle size) | | |
| Column | 35° C. | | |
| temperature | | | |
| Mobile phase | A: CO₂, bone-dry, without dip-tube (Air Gas UN1013) | | |
| | B: MeOH | | |
| Flow | 3 mL/min | | |
| Gradient | 0 min | 1% B | 2 mL/min |
| | 0.05 min | 1% B | 3 mL/min |
| | 1 min | 3% B | 3 mL/min |
| | 6 min | 30% B | 3 mL/min |
| | 11 min | 30% B | 3 mL/min |
| | 11.5 min | 1% B | 3 mL/min |
| | 11.95 min | 1% B | 2 mL/min |
| | 12 min | 1% B | 2 mL/min |

| | |
|---|---|
| Pressure limit | 20-550 bar |

### Example 1

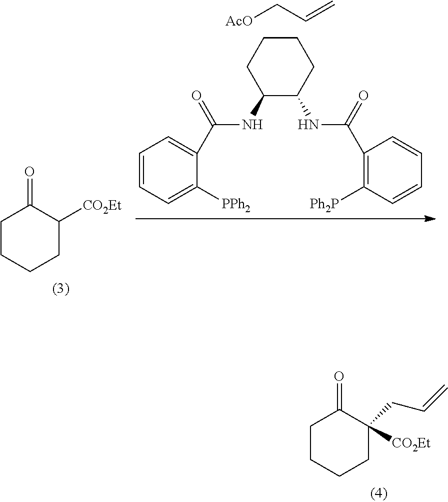

A dry and clean reactor is charged with toluene (234 L) under nitrogen (note: 2.5V toluene in total for this reaction). Water (1.56 kg, 85.5 mol, keep H₂O: Pd=160:1) is added followed by rinsing the charging line with 1,1,3,3-tetramethylguanidine (175.5 kg, 1527.6 mol, 2.0 equiv.) under nitrogen and then toluene (13 L). 3 (130.0 kg, 763.8 mol) is added under nitrogen followed by rinsing with toluene (13 L). Allyl acetate (98.8 kg, 992.9 mol, 1.3 equiv.) is added under nitrogen and rinsed with toluene (13 L). Under agitation, the mixture is cooled to 10° C. in 0.5 h. The batch is degassed by sparging the solution with nitrogen for ˜30 min. (S,S)-DACH-Ph Trost ligand (0.429 kg, 0.619 mol, 0.081 mol %) in degassed toluene (13 L) (note: keep Pd: ligand=1: 1.15) is added followed by rinsing with degassed toluene (13 L). Allylpalladium(II) chloride dimer (97.5 g, 0.267 mol, 0.035 mol %) in degassed toluene (13 L) is added followed by rinsing with degassed toluene (13 L). The batch is kept at 10-15° C. at least 8 h. After the reaction is complete by HPLC, a solution of N-acetyl-L-cysteine (3.9 kg, 22.9 mol, 0.03 equiv.) in water (260 L) below 25° C. is added. The resulting solution is warmed to 20-25° C. and kept at 20-25° C. at least for 1 h. After phase cut to discard the bottom aqueous layer, 10 wt % NH₄Cl aqueous solution (260 L) is added. After the mixture is agitated for 10 min, the bottom aqueous layer is drained. The organic phase is further washed with water (130 L). The organic layer is filtered through a very short pad of Celite and the reactor and Celite bed is rinsed with toluene (65 L). The filtrate is charged into a clean reactor and then toluene is distilled off under vacuum at 40-50° C. The crude product is directly used for the next step or the product is drained into a container with the help of minimum amount of toluene (65 L) and stored at 20-23° C. 150 kg of 4 is usually obtained as a light yellow oil in 96% yield with ≥ 90:10 enantiomeric ratio (Chiral method I). 1H NMR (500 MHZ, CDCl₃): δ 5.75 (ddt, J=14.8, 9.4, 7.5 Hz, 1H), 5.06-5.00 (m, 2H), 4.19 (q, J=7.1 Hz, 2H), 2.61 (dd, J=13.9, 7.1 Hz, 1H), 2.51-2.43 (m, 3H), 2.33 (dd, J=13.9, 7.9 Hz, 1H), 2.03-1.98 (m, 1H), 1.78-1.60 (m, 3H), 1.50-1.42 (m, 1H), 1.25 (t, J=7.1 Hz, 3H). 13C NMR (125 MHz, CDCl₃): δ 207.7, 171.6, 133.5, 118.4, 61.3, 61.0, 41.3, 39.4, 35.9, 27.7, 22.6, 14.3. ESI-MS: m/z 211 [M+H]⁺.

### Example 2

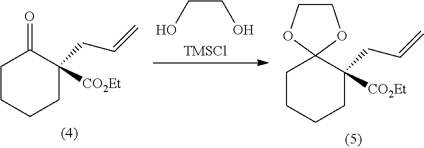

To the reactor containing 4 (150 kg, 713.4 mol) from step 1 (less than 1V toluene if used) is added ethylene glycol (600 L) to give a yellow biphasic mixture. After the mixture is cooled to 10-15° C., TMSCl (193.5 kg, 1783.5 mol, 2.5 equiv.) is added over not less than 15 min, at a rate to maintain the internal temperature between 20-30° C. (orange biphasic mixture obtained). Sufficient agitation is needed to achieve mixing. After the batch is kept at 20-25° C. for 2 h, agitation is stopped and kept for at least 15 min at 20-25° C. The batch is cooled to 0-5° C. NaOH (96 kg, 1854.8 mol, 2.6 equiv.) in water (600 L) is added at a rate to maintain the internal temperature below 20° C. (light yellow cloudy biphasic mixture obtained). Toluene (300 L) is added and then the batch is agitated for 10 min. After phase cut to drain the bottom aqueous layer (note: some precipitate may form at interphase), the organic layer is washed with water (300 L) two times. The organic phase is filtered through a short pad of Celite to remove insoluble solids/interphase. The organic solution is charged into a clean and dry reactor and then the solvent is distilled off at 40-50° C. to a minimum stirrable volume. The crude product 5 (189 kg, 95.2 wt %, 100% yield) is drained to a container with the help of minimum amount of toluene.

1H NMR (500 MHZ, CDCl₃): δ 5.65 (ddt, J=14.7, 8.1, 6.6 Hz, 1H), 5.07-4.98 (m, 2H), 4.20-4.10 (m, 2H), 3.97-3.88 (m, 4H), 2.81 (dd, J=13.9, 6.6 Hz, 1H), 2.35 (dd, J=13.9, 8.1 Hz, 1H), 2.04-1.98 (m, 1H), 1.75-1.35 (m, 7H), 1.26 (t, J=7.1 Hz, 3H). 13C NMR (125 MHz, CDCl₃): δ 173.7, 134.3, 117.6, 110.9, 65.0, 64.7, 60.5, 54.6, 36.2, 32.3, 30.3, 23.3, 20.9, 14.4. ESI-MS: m/z 255 [M+H]⁺.

### Example 3

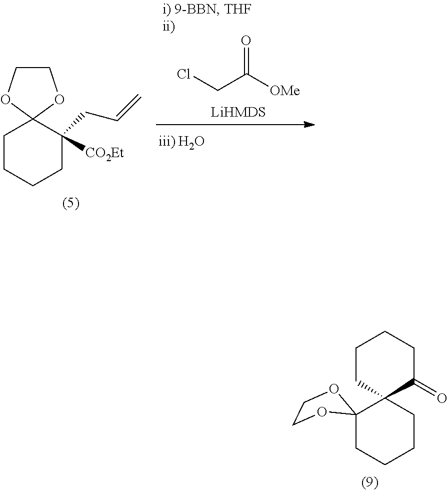 [SMILES: C=CC[C@]1(C(=O)OCC)CCCCC12OCCO2 ; COC(=O)CCl ; O=C1CCCC[C@@]12CCCCC21OCCO1]

To a dry and clean reactor is added 9-BBN (688.5 kg, 401 mol, 1.2 equiv.) under nitrogen. The solution is cooled to 0-5° C. to obtain a slurry. 5 (85.0 kg, 334.2 mol) from step 2 is added at 0-5° C. and rinsed with THF (40 L). The mixture is warmed to 20-23° C. in 1 h and kept at 20-23 ºC for not less than 1 h. After that the mixture is cooled to −45 to −40° C., methyl chloroacetate (69.6 kg, 1.3 equiv.) is added in one portion followed by dropwise addition of LiHMDS (909.5 kg, 1102.9 mol) while keeping temperature below −35° C. The batch is then warmed to 20-23° ° C. in 1 h and then kept at 20-23° C. at least for 18 h. ˜12-13 V solvent is removed by distillation under vacuum with heating (35°) C.. EtOH (255 kg) is added followed by a solution of NaOH (13.4 kg) in H₂O (212.5 L). The mixture is heated at reflux (at 66-70° C.) for at least 14 h.

After that ˜5-6 V solvent is removed by distillation at reflux, the batch is cooled to 20-25° C. and then filtered through a short pad of Celite to remove insoluble material and rinsed with heptane (160 L). ˜5-6 V solvent (or most of the residual THF and ethanol) is distilled under vacuum at 40-50° C. The batch is cooled to 20-25° C. After that, water (255 L) is added, the crude product is extracted twice with heptane (2364.8 kg). The combined heptane layers are washed once with water (85 L). After solvent removal by distillation under vacuum at 40-50° C., the crude product (52.7 kg, 87.5 wt %) is obtained as a yellow oil in 52.6% assay yield with ≥90:10 enantiomeric ratio (Chiral method II). The crude product 9 is used for next step directly.

1H NMR (500 MHZ, CDCl₃): δ 4.01-3.82 (m, 4H), 2.50-2.44 (m, 1H), 2.382.34 (m, 1H), 2.28-2.22 (m, 1H), 2.11-2.05 (m, 1H), 2.01-1.95 (m, 1H), 1.92-1.86 (m, 1H), 1.81-1.58 (m, 6H), 1.54-1.43 (m, 3H), 1.27-1.18 (m, 1H). ESI-MS: m/z 225 [M+H]⁺.

### Example 3a

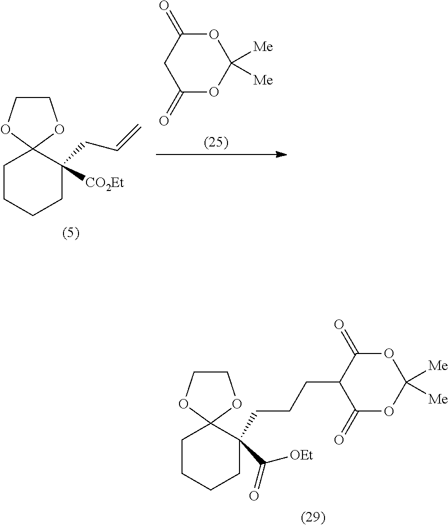

To a clean reactor under dry N2 atmosphere is charged Meldrum's acid (25) (11.33 g, 78.64 mmol, 2 eq), borane 28 (0.78 g, 10 mol %) and acetonitrile (30 mL, 3 V). The mixture is then agitated to form a clear solution. 5 is charged (10 g, 39.3 mmol, 1 eq) followed by additional acetonitrile (5 mL, 0.5 V). To the mixture is then charged photocatalyst 26 (0.62 g, 0.79 mmol, 2 mol %) and the mixture is agitated until 26 is completely dissolved. Triphenylsilanethiol (27) (0.58 g, 1.97 mmol, 10 mol %) and acetonitrile (15 mL, 1.5 V) are charged. The mixture is then agitated to form a yellow homogeneous solution. Photochemistry reaction is then performed using common commercial photoreactors or customized reactors that are described in literature (Chem. Rev. 2022, 122, 2, 2752-2906). The photoreactor is preheated to 50° C. and LED is turned on. The reaction mixture is then introduced into a flow photoreactor with a suitable dosing pump to maintain a residence time of 50 min to 200 min. Product solution containing 29 is collected simultaneously. Crude product solution is used for next step without purification.

29: 1H NMR (500 MHZ, CDCl₃): δ 4.14-4.07 (m, 2H), 3.92-3.82 (m, 4H), 3.52 (t, 1H), 2.05-1.93 (m, 4H), 1.73 (d, 6H), 1.67-1.57 (m, 4H), 1.54-1.29 (m, 5H), 1.24-1.21 (m, 4H). 13C NMR (500 MHz, CDCl₃): δ 173.9, 165.4, 165.3, 110.9, 104.7, 64.7, 64.5, 60.4, 54.5, 45.8, 32.1, 31.1, 30.2, 28.4, 26.8, 26.7, 23.0, 21.7, 20.8, 14.1

ESI-MS: m/z 421 [M+Na]⁺.

### Example 3b

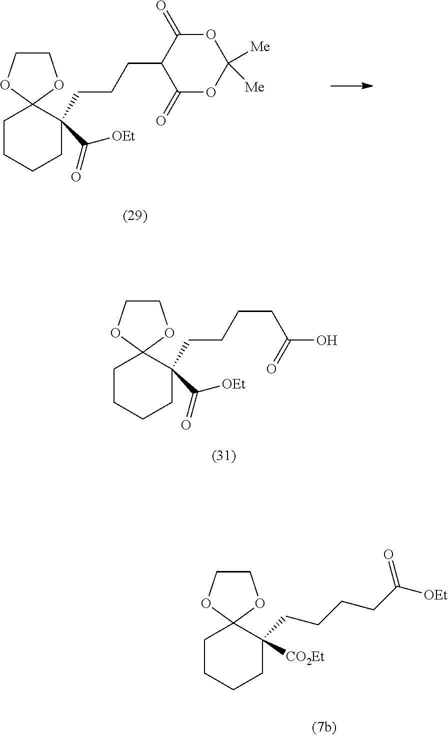 [SMILES: CCOC(=O)[C@@]1(CCCC2C(=O)OC(C)(C)OC2=O)CCCCC12OCCO2 ; CCOC(=O)[C@@]1(CCCCC(=O)O)CCCCC12OCCO2 ; CCOC(=O)CCCC[C@]1(C(=O)OCC)CCCCC12OCCO2]

A clean reactor is charged with crude solution of 29 (100 g, 40.7 mmol, 1 eq) produced from the flow process and imidazole (6.92 g, 101.6 mmol, 2.5 eq). The mixture is agitated at internal temperature of 80-85° C. for 3.5 hours. The reaction is cooled to 70° C. and water is charged (100 mL, 1V). Reaction temperature maintained at an internal temperature of 65° C. for 3 hours. The mixture is then agitated at 25° C. for another 16 hours.

The reaction is worked up following the process below; 4 M NaOH solution (50 mL, 200 mmol, 5 eq) is charged and the mixture is agitated for 30 minutes. MTBE is charged and the mixture is agitated. After Phase cut the Methyl tert-butyl ether layers is removed and the aqueous layer is filtered through a filter paper. To the aqueous phase 2 M HCl solution (5.2 V) is charged to adjust pH between 3.5 to 5.0, while internal temperature maintains at 25° C. EtOAc is charged to the acidic aqueous mixture and the mixture is agitated. After Phase cut EtOAc phases are collected. Concentration of EtOAc gives the crude product 31, which is used directly for next steps.

31: 1H NMR (500 MHZ, CDCl₃): δ 4.22-4.11 (m, 2H), 3.97-3.86 (m, 4H), 2.36 (t, 2H), 2.11-2.00 (m, 2H), 1.76-1.50 (m, 9H), 1.41-1.32 (m, 2H), 1.28-1.26 (t, 3H), 1.16-1.07 (m, 1H). 13C NMR (500 MHz, CDCl₃): δ 178.96, 174.04, 111.07, 64.77, 64.59, 60.35, 54.41, 33.69, 32.06, 30.68, 29.64, 25.17, 23.93, 23.11, 20.72, 14.25.

ESI-MS 315 [M+H]⁺

To a clean reactor under nitrogen CDI is charged (8.67 g, 53.55 mmol, 1.5 eq), followed by toluene (22.4 mL, 2V). In a separated mixing vessel, a solution of 31 (11.2 g, 35.63 mmol, 1 eq) is prepared in toluene (11.3 mL, 1V). The solution of 31 in toluene is then slowly dosed into the reactor containing the CDI/toluene slurry. The mixture is heated to 55 to 60° C. for about 1 hour. After that, ethanol is charged (11.3 mL, 1V) and the mixture is heating for 2 hours at 75-80° C. The reaction is cooled to 20 to 25° C. The organic phase is washed with water (22.4 mL, 2V), 1 M HCl (22.4 mL, 2V) and then water (22.4 mL, 2V). The organic phase is filtered through a pad of celite. The filter is then rinsed with toluene (5.6 mL, 0.5V). Toluene fractions are combined and distilled at 30 to 60° C. under vacuum to afford the desired product 7b in 92-95% yield.

7b: 1H NMR (500 MHZ, CDCl₃): δ 4.25-4.05 (m, 4H), 3.95-3.86 (m, 4H), 2.30-2.26 (m, 2H), 2.06-1.98 (m, 2H), 1.75-1.48 (m, 9H), 1.40-1.22 (m, 8H), 1.14-1.02 (m, 1H).

### Example 3c

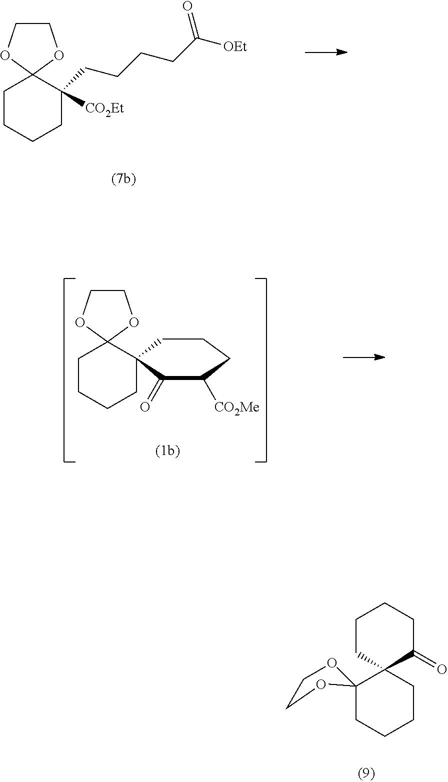

A dry and clean reactor with LiHMDS (67.2 mL, 2.3 eq, 1M in THF) is cooled to −5 to 0° C., 7b (10.0 g, 1 eq) in THF is charged slowly while maintaining internal temperature below 5° C. and the mixture is held at 5° C. for 30 minutes. EtOH (25 mL, 2.5 V) is charged, and the mixture is heated to 66-70° C. for 14 hours. Then 5-6 V solvent is removed by vacuum distillation and the remaining solution is filtered through a pad of celite. The celite pad is rinsed with MTBE (20 mL, 2V). The organic solutions are extracted with MTBE and water. The combined MTBE fraction is then filtered through a pad of celite. After vacuum distillation at 30-60° C., desired product 9 is obtained in 90-95% yield. The analytical data is identical to 9 obtained in example 3.

### Example 4

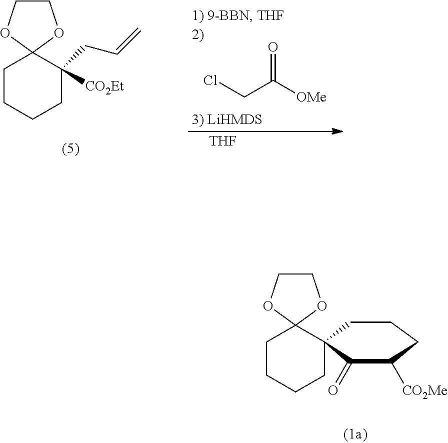

A dry and clean reactor is charged with 9-BBN (387 mL, 193.5 mmol, 1.2 equiv., 0.5 M in THF) under nitrogen. The solution is cooled to 0-5° C. to obtain a slurry. 5 (41.0 g, 161.2 mmol) is added at 0-5° C. and rinsed with THF (20.5 mL). The mixture is warmed to 20-23° C. in 1 h and kept at 20-23° C. for not less than 1 h. After the mixture is cooled to −45 to −40° C., methyl chloroacetate is added in one portion followed by dropwise addition of LiHMDS (355 mL, 532.0 mmol, 3.3 equiv.) while keeping temperature below −35° C. The batch is then warmed to 20-23° C. in 1 h and then kept at 20-23° C. at least for 18 h. The batch is cooled to 5-10° C., AcOH (30.4 mL, 3.3 equiv.) is added below 20° C. followed by water (41 mL) below 20° C. AcOH (30.4 mL, 3.3 equiv.) is added below 20° C. to reach PH ˜6-7. ˜15-16 V of THF is removed under vacuum below 35° C. MTBE (246 mL) and water (205 mL) are added. After phase cut to discard the bottom aqueous layer, the mixture is cooled to 0-5° C., a solution of sodium percarbonate (37.2 g, 322.4 mmol, 2.0 equiv.) in water (320 mL) is added below 20° C. After 1 h at 20-23° ° C., 20 wt % sodium sulfite solution (31 mL) is added. After 15 min at 20-23° C., the bottom aqueous layer is separated and discarded. The organic layer is washed with 5 wt % ammonium chloride solution (123 mL) and water (328 mL). The organic layer is treated with 5% activated carbon for 30 min prior to filtration. After ˜4-5 V of solvent is removed under vacuum below 35° C. the crude product 1a (80% yield; [M+H]⁺=283) is obtained as orange-brown oil.

### Example 5

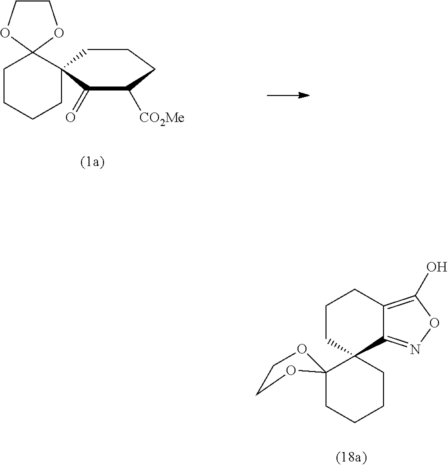

A reactor is charged with 1a (45.5 g, 161.2 mmol), ethanol (91.0 mL), NaOAc (39.7 g, 483.6 mmol, 3.0 equiv.), water (45.5 mL) and NH₂OH·HCl (33.6 g, 483.6 mmol, 3.0 equiv.). The mixture is heated at 73-78° C. for not less than 16 h. After the batch is cooled to 20-23° C., water (227.6 mL) is added over 0.5 h. Then MTBE (136.5 mL) is added over 0.5 h followed by heptane (113.8 mL) over 1 h. After 0.5 h at 20-23° C., the solid is collected by filtration. The solid is washed successively with MTBE (45 mL) and water (91.0 mL). The solid is dried under vacuum to give the product 18a as an off-white solid (18.27 g, 93.2 wt %) in 40% yield.

1H NMR (500 MHZ, DMSO-d6): δ 11.48 (br s, 1H), 4.04 (q, J=6.0 Hz, 1H), 3.89-3.80 (m, 2H), 3.60 (q, J=6.8 Hz, 1H), 2.10-1.95 (m, 2H), 1.92-1.76 (m, 4H), 1.69-1.39 (m, 8H). ESI-MS: m/z 266 [M+H]⁺.

### Example 6

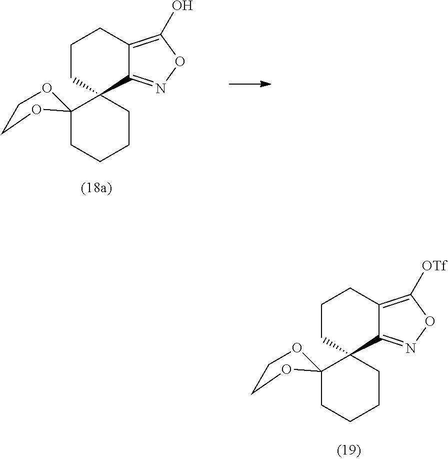 [SMILES: Oc1onc2c1CCC[C@@]21CCCCC12OCCO2 ; O=S(=O)(Oc1onc2c1CCC[C@@]21CCCCC12OCCO2)C(F)(F)F]

A clean reactor is charged with 18a (100.0 g, 376.9 mmol, 1.0 equiv.), and K₃PO₄ (240.0 g, 1130.8 mmol, 3.0 equiv.) in water (499.0 g, 500.0 mL) and toluene (432.5 g, 500.0 mL). The bi-phase mixture is agitated to sufficient mixing. After the mixture is cooled to 0˜5° C., Tf₂O (186.0 g, 110.9 mL, 659.6 mmol, 1.750 equiv.) is added with a syringe pump over 2 h below 5° C. After phase cut, the organic layer is filtered through a Celite bed with Na₂SO₄. After rinsing with toluene (50 mL), the crude product 19 (149.8 g, 100% yield) is used for the next step directly.

1H NMR (500 MHZ, CDCl₃): δ 3.95-3.91 (m, 3H), 3.77-3.74 (m, 1H), 2.51-2.44 (m, 2H), 2.16-1.80 (m, 4H), 1.77-1.48 (m, 8H). ESI-MS: m/z 398 [M+H]⁺.

### Example 7

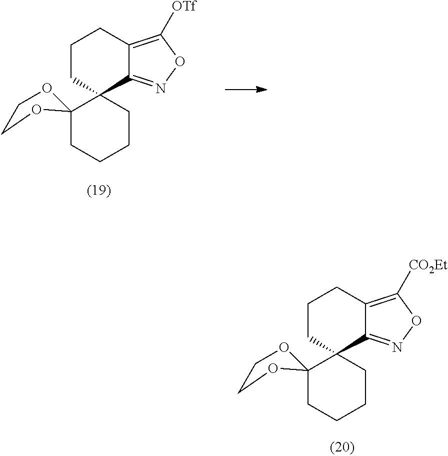 [SMILES: O=S(=O)(Oc1onc2c1CCC[C@@]21CCCCC12OCCO2)C(F)(F)F ; CCOC(=O)c1onc2c1CCC[C@@]21CCCCC12OCCO2]

A dry and clean autoclave reactor is charged with 19 (750 g, 1.89 mol, 1 equiv.), Pd(OAc)₂ (8.48 g, 37.7 mmol, 0.02 equiv.), rac-BINAP (23.5 g, 37.7 mmol, 0.02 equiv), 2-MeTHF (3 L), EtOH (870 g, 18.9 mol, 10 equiv.) and DIPEA (293 g, 2.26 mol, 1.2 equiv.). The reactor is purged with nitrogen (100 psi) two times and then purged with CO (100 psi) two times. The reactor is pressurized to 200 psi CO and heated at 55-60° C. for not less than 12 h. The mixture is transferred to a reactor and the autoclave reactor is rinsed with 2-MeTHF (0.75 L) into the reactor. The mixture is washed with water (3.75 L). After filtration through a short Celite pad, the solvent is removed by vacuum distillation to give the crude product 20 (531.9 g, 87.7% yield) which is used for the next step without purification.

1H NMR (400 MHZ, CDCl₃): δ 4.38 (q, J=7.1 Hz, 2H), 3.95-3.85 (m, 3H), 3.76-3.73 (m, 1H), 2.85 (dt, J=17.5, 5.5 Hz, 1H), 2.64 (ddd, J=17.5, 9.6, 6.0 Hz, 1H), 2.22-2.14 (m, 1H), 2.04-1.88 (m, 3H), 1.78-1.45 (m, 8H), 1.37 (t, J=7.1 Hz, 3H). ESI-MS: m/z 322 [M+H]⁺.

### Example 8

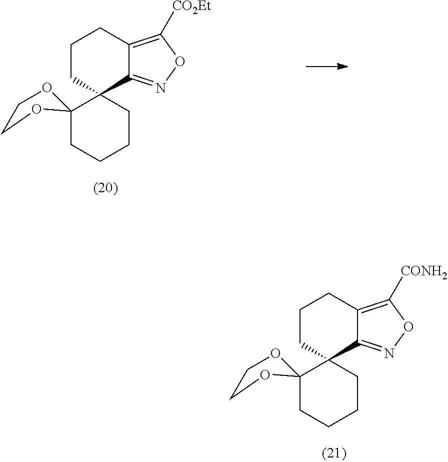 [SMILES: CCOC(=O)c1onc2c1CCC[C@@]21CCCCC12OCCO2 ; NC(=O)c1onc2c1CCC[C@@]21CCCCC12OCCO2]

A dry and clean reactor is charged with 20 (482.0 g, 1.5 mol, 1 equiv.) and EtOH (3 V) and vacuum distilled ˜3 V to remove residual 2-MeTHF from the previous carbonylation step. EtOH (1.45 L) and NH₄OH (1.93 L) are added. The mixture is kept at 20-25° C. for not less than 15 h. Water (1.69 L) is added over 30 min. After 30 min at 20-25° C., the solid is collected and washed with 1:2 EtOH/water (0.96 L) and water (0.48 L). The solid is slurried in 1:1 MTBE/hexane (0.96 L) for 1 h. The solid is collected by filtration and dried under vacuum at 40-45° C. overnight to give the product 21 (332.4 g, 75.8% yield, water content ≤0.5% based on Karl Fischer titration) as a tan solid.

1H NMR (500 MHZ, DMSO-d6): δ 8.05 (s, 1H), 7.78 (s, 1H), 3.94-3.72 (m, 4H), 2.78 (dt, J=17.1, 5.0 Hz, 1H), 2.54-2.48 (m, 1H), 2.20-2.14 (m, 1H), 1.93-1.78 (m, 3H), 1.70-1.42 (m, 8H). ESI-MS: m/z 293 [M+H]⁺.

### Example 9

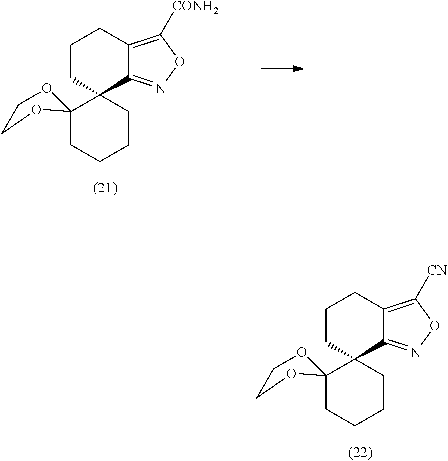 [SMILES: NC(=O)c1onc2c1CCC[C@@]21CCCCC12OCCO2 ; N#Cc1onc2c1CCC[C@@]21CCCCC12OCCO2]

A dry and clean reactor is charged with 21 (383 g, 86.7 wt %, 1.137 mol, 1 equiv.), MeCN (1.15 L) and pyridine (216 g, 0.19 L, 2.4 equiv.). After the mixture is cooled to 0-5° C., trifluoroacetic anhydride (287 g, 1.36 mol, 1.2 equiv.) is added below 5° C. After 5 min at 0-5° C., water (1.54 L) is added below 15° C. The product is extracted with MTBE (1.92 L) and washed with 5% sodium bicarbonate solution (1.15 L). The organic layer is filtered through silica gel pad (380 g) and rinsed with MTBE (0.58 L). After resolvent removal by distillation under vacuum, the product 22 (421.8 g, 97.8% yield) is obtained as an orange-brown oil.

1H NMR (500 MHZ, CDCl₃): δ 3.98-3.85 (m, 3H), 3.80-3.75 (m, 1H), 2.72 (dt, J=17.0, 5.2 Hz, 1H), 2.60 (ddd, J=17.0, 9.5, 5.8 Hz, 1H), 2.20-2.12 (m, 1H), 2.07-1.94 (m, 3H), 1.82-1.48 (m, 8H). ESI-MS: m/z 275 [M+H]⁺.

### Example 10

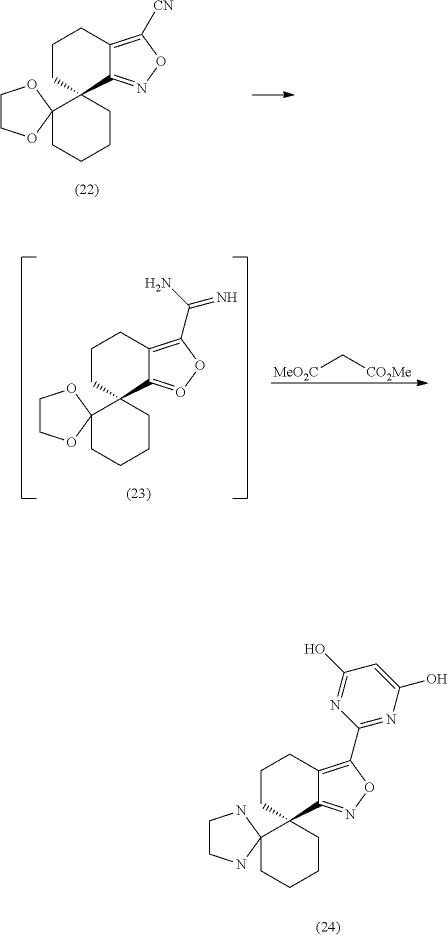

A dry flask is charged with crude 22 (265 g, 72.3 wt %, 698.4 mmol) in MeOH (1590 mL) and cat. NaOMe (8.0 mL, 25% in MeOH, 34.9 mmol). The mixture is stirred at rt for 1 h to achieve >99% conversion. After solid NH₄Cl (52.0 g, 977.8 mmol, 1.4 equiv.) is added, the resulting mixture is stirred at rt to achieve >95% conversion (if not, more NH₄Cl is added). After dimethyl malonate (168 g, 1047.7 mmol, 1.5 equiv.) is added at rt, NaOMe (377 g, 25% in MeOH, 2.5 equiv.) is added. The resulting mixture is heated to reflux for 4 h to achieve >95% conversion. After the mixture is cooled to 23° C., water (795 mL) is added followed by addition of 6N HCl (349 mL) slowly below 20° C. to reach pH ˜3. To the slurry is added MTBE (530 mL). After 1 h at rt, the solid is collected by filtration, washed with 3V water (796 mL) and MTBE (530 mL) to give the product 24 (178 g) as an off-white solid with >98A % and 71% crude yield. The crude product is used for next step directly.

1H NMR (500 MHZ, CDCl₃): δ 5.82 (s, 1H), 3.96-3.74 (m, 4H), 2.74-2.70 (m, 1H), 2.62-2.59 (m, 1H), 2.22-2.10 (m, 1H), 2.12-1.90 (m, 3H), 1.80-1.48 (m, 8H). ESI-MS: m/z 360 [M+H]⁺.

### Example 11

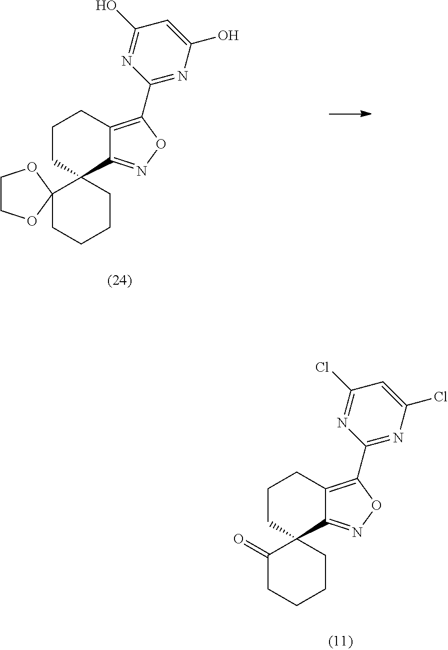 [SMILES: Oc1cc(O)nc(-c2onc3c2CCC[C@@]32CCCCC23OCCO3)n1 ; O=C1CCCC[C@@]12CCCc1c2noc1-c1nc(Cl)cc(Cl)n1]

A dry flask is charged with 24 (80.0 g, 253.7 mmol), DMAP (4.0 g), tetramethyl ammonium chloride (4.0 g), and POCl₃ (400 mL). The mixture is heated at 80° C. for 1.5 h to achieve >99% conv. POCl₃ is removed under vacuum to get a thick light-yellow slurry. MTBE (160 mL) is added. Then the mixture is cooled to 5° C. Water (800 mL) is slowly added. The resulting white slurry is stirred at 23° C. for 1 h. The solid is collected by filtration and then washed successively with water (480 mL) and MTBE (160 mL). After drying under vacuum at 60° ° C. overnight, 84.3 g of the product 11 are isolated as a white solid in >99 purity % and ˜93% yield.

1H NMR (600 MHZ, DMSO-d6): δ 8.05 (s, 1H), 2.96-2.91 (m, 1H), 2.76-2.69 (m, 2H), 2.53-2.48 (m, 2H), 2.37-2.34 (m, 1H), 1.97-1.96 (m, 2H), 1.88-1.82 (m, 4H), 1.70-1.61 (m, 1H), 1.52-1.41 (m, 1H). 13C NMR (125 MHz, DMSO-d6): δ 209.8, 164.3,161.4, 157.3, 155.7, 120.8, 120.2, 50.3, 38.1, 37.5, 31.0, 26.6, 20.7,19.9, 18.0. ESI-MS: m/z 353 [M+H]⁺.

### Example 12

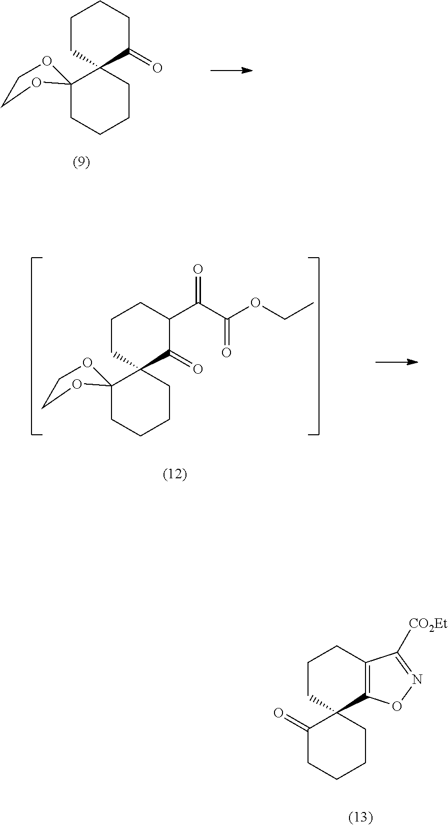

A dry and clean reactor is charged with LiHMDS (1 M in THF) (406.4 kg, 456.1 mol, 1.1 equiv.). The solution is cooled to 0-5° C., crude 9 (93.0 kg, 414.6 mol) is added below 5° C. and rinsed with THF (46.5 kg) to aid transfer. After 30 min at 0-5° C., diethyl oxalate (72.5 kg, 497.5 mol, 1.2 equiv.) is added below 5° C. After the mixture is warmed to 20-25° C. in 1 h, the mixture is kept at 20-25° C. for not less than 3 h. After the batch is cooled to 10-15° C., cooled HCl solution [prepare by adding acetyl chloride (73.6 kg, 932.9 mol, 2.25 equiv.) to EtOH (293.9 kg) at 0-5° C.] is added to the batch below 25° C. to reach final pH ˜6-7 of the yellow slurry. Solid NH₂OH. HCl (28.8 kg, 414.4 mol, 1.05 equiv.) is added in one portion and the resulting mixture is heated to reflux 66-70° C. for 6-10 h. After that 5V of solvent is removed by distillation at reflux 66-70° C. EtOH (73.5 kg) is used to remove residual THF. Water (372.0 kg) and EtOH (293.9 kg) are added. After 3-6 h at 70-75° C., the mixture is cooled to 30-35° C. 0.5-1% 13 crystals are seeded. After 2-4 h at 30-35° C., heptane (63.2 kg) is added in not less than 1 h. After 60 min at 20-25° C., water (279.0 kg) is added over 4-6 h. After 1 h at 20-25° C., the solid is collected and washed with 1:2 EtOH/water (51.2 kg EtOH and 130.2 kg water) and then heptane (63.2 kg) two times. The solid is dried under vacuum under nitrogen stream to give the product 13 (93.0 kg) with 65% yield.

1H NMR (500 MHZ, CDCl₃): δ 4.42 (q, J=7.1 Hz, 2H), 2.73 (dt, J=16.8, 5.1 Hz, 1H), 2.64 (dt, J=14.3, 6.0 Hz, 1H), 2.60-2.51 (m, 2H), 2.43-2.30 (m, 2H), 2.09-1.96 (m, 3H), 1.91-1.81 (m, 3H), 1.76-1.67 (m, 1H), 1.65-1.58 (m, 1H), 1.40 (t, J=7.1 Hz, 3H). ESI-MS: m/z 278 [M+H]⁺.

### Example 13

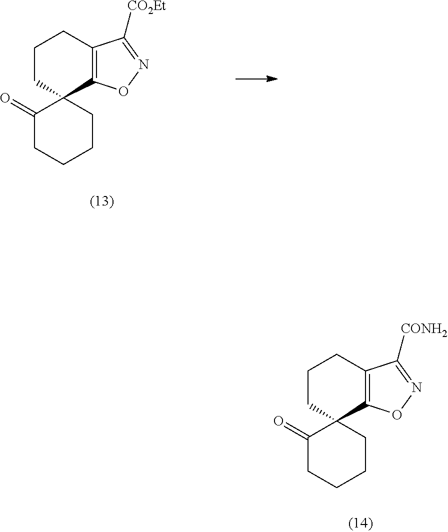 [SMILES: CCOC(=O)c1noc2c1CCC[C@@]21CCCCC1=O ; NC(=O)c1noc2c1CCC[C@@]21CCCCC1=O]

A dry and clean reactor is charged with 13 (72.0 kg, 259.6 mol), EtOH (56.9 kg) and NH₄OH (aq) (280.8 kg). The mixture was kept at 20-25° C. for not less than 16 h. After water (144.0 kg) is added over 30 min, the slurry is kept at 20-25° C. for 30 min. The solid is collected by filtration, washed with 1:3 EtOH/water (28.5 kg EtOH and 108 kg water) and then heptane (97.9 kg). After drying, under vacuum over 1 h at 23° C., the solid was dried under vacuum at 50-55° C. overnight to give the product 14 (61.4 kg, 87.2% yield, enantiomeric ratio ≥ 95:5 (254 nm), water content ≤0.5% based on Karl Fischer titration).

A dry and clean reactor is charged with crude 14 (60.0 kg, 1.0 equiv.), 1,4-dioxane (240.0 kg) and activated carbon (3.0 kg, 5 wt %). The mixture is stirred at 55-65° C. for 2-4 h. After filtration at high temperature (55˜65°) C., the filter cake is washed with 1,4-dioxane (33.0 kg). The filtrate is transferred into a clean reactor. The temperature is adjusted to 45-55° C. and stirred at 45-55° C. for 1-2 h. Water (240.0 kg) is added over 2 h. The temperature is adjusted to 45-55° C. and stirred at 45-55° C. for 1-2 h. The mixture is cooled down to 35-45° C. and stirred at 35˜45° C. for 2-4 h. Water (87.0 kg) is added over 4 h. The mixture is cooled down to 15-25° C. and stirred at 15-25° C. for 12-14 h. The solid is collected by a centrifuge, washed with water (120.0 kg) and dried under vacuum at 50-55° C. overnight to give the product 14 (44.8 kg, 71% yield) as a light yellow to off-white solid.

1H NMR (500 MHZ, DMSO-d6): δ 7.99 (s, 1H), 7.71 (s, 1H), 2.80-2.69 (m, 1H), 2.60-2.53 (m, 1H), 2.50-2.42 (m, 1H), 2.40-2.28 (m, 2H), 2.26-2.18 (m, 1H), 2.05-1.70 (m, 7H), 1.48-1.39 (m, 1H). ESI-MS: m/z 249 [M+H]⁺.

### Example 14

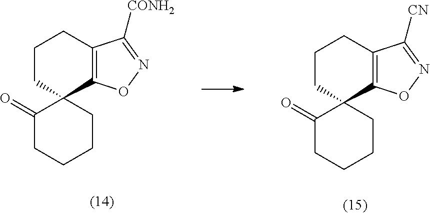 [SMILES: NC(=O)c1noc2c1CCC[C@@]21CCCCC1=O ; N#Cc1noc2c1CCC[C@@]21CCCCC1=O]

A dry and clean reactor is charged with 14 (40.0 kg, 161.1 mol), MeCN (96.0 kg) and pyridine (30.8 kg, 386.6 mol, 2.4 equiv.). After the mixture is cooled to 0-5° C., TFAA (40.8 kg, 193.3 mol, 1.2 equiv.) is added slowly below 5° C. After 5 min at 0-5° C., water (120.0 kg) is added over 30 min at 0-5° C. and seeded with 0.5% 15 crystals. After 15 min at 0-5° C., water (120.0 kg) is added over 30 min. After 30 min at 0-5° C. for 30 min, the solid is collected by filtration, washed with 1:3 MeCN/water (15.6 acetonitrile and 60.0 kg water) and then water (80.0 kg). The solid is dried under vacuum to give the crude product (33.0 kg, 93.6% yield) as a tan solid.

A dry and clean reactor is charged with crude 15 (32.5 kg, 1.0 equiv.) and MTBE (48.1 kg), the slurry is agitated at 20-25° C. for 30 min. Heptane (132.6 kg) is added over 1 h. After 30 min at 20-25° C., the solid is collected, dried under vacuum to give the product 15 (26.6 kg, 82.0% yield) as a white solid with >99:1 enantiomeric ratio (254 nm, chiral method III) and >98% purity (220 nm).

1H NMR (500 MHZ, DMSO-d6): δ 2.83-2.73 (m, 1H), 2.60-2.40 (m, 3H), 2.34-2.20 (m, 2H), 2.06-1.75 (m, 7H), 1.53-1.43 (m, 1H). ESI-MS: m/z 231 [M+H]⁺.

### Example 15

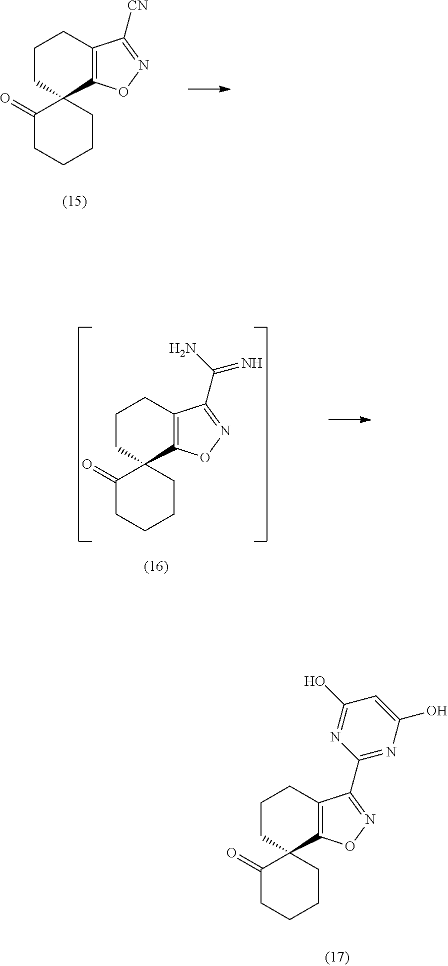

To a stirred solution of 15 (25.0 g, 108.6 mmol, 1.0 equiv.) in MeOH (150 mL), is added NaOMe (30% in MeOH, 4.89 g, 27.1 mmol, 0.25 equiv.) and the resulting mixture is stirred for 2 h at rt. Then NH₄Cl (6.39 g, 119.4 mmol, 1.1 equiv.) is added and the mixture is stirred for 16 h at rt. After complete conversion to the desired amidine, the mixture is filtered through a Celite bed and concentrated. The residue is dissolved in DMF (125 mL), 1,8-diazabicyclo[5.4.0]undec-7-ene (32.3 g, 212.3 mmol, 2.1 equiv.) and diethyl malonate (13.4 g, 101.1 mmol, 1.0 equiv.) are added at 0° C. and the resulting mixture is stirred for 16 h at 90° C. After complete conversion, ice cold water is added, the mixture is acidified with IN HCl and the precipitate is collected by filtration. The precipitate is dried under reduced pressure yielding crude 17 (,[M+H]=316) which is used for the next step without purification.

### Example 16

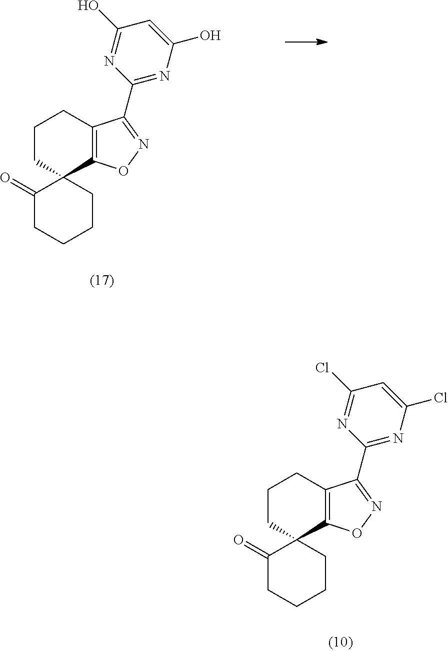 [SMILES: O=C1CCCC[C@@]12CCCc1c(-c3nc(O)cc(O)n3)noc12 ; O=C1CCCC[C@@]12CCCc1c(-c3nc(Cl)cc(Cl)n3)noc12]

17 (10.0 g, 30.1 mmol, 1.0 equiv.) and POCl₃ (48.0 g, 310.0 mmol, 10.3 equiv.) are combined at 0° C. and stirred for 5 min. DIPEA (8.2 g, 63.2 mmol, 2.1 equiv.) is added and the resulting mixture is stirred for 3 h at 80° ° C. After complete conversion ice cold water (1 L) is slowly added to the mixture at 0° C. and afterwards the mixture allowed to reach rt and stirred for 1 h. The precipitate is collected by filtration, washed with water and hexane, and dried under vacuum to yield 10 (, [M+H]=352/354). The crude product is used for the next step without purification.

What we claim:

1. A compound of formula (1) 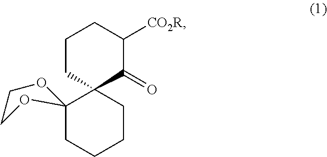
wherein R is selected from the group consisting of linear, branched, or cyclic alkyl groups, and aryl groups, as well as combinations thereof.

2. A process for the preparation of a compound of formula (1) according to claim 1, wherein the process comprises reacting a compound of the following formula (5) in a one-pot reaction:

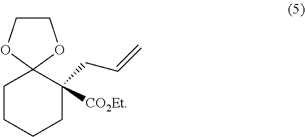 [SMILES: C=CC[C@]1(C(=O)OCC)CCCCC12OCCO2]

3. The process according to claim 2, where the one-pot reaction from a compound of formula (5) to a compound of formula (1) comprises at least 3 transformations: hydroboration, alkylation and Dieckmann Condensation.

4. The process according to claim 3, wherein the hydroboration is carried out in the presence of 9-BBN.

5. The process according to claim 3, wherein the alkylation is carried out in the presence of an α-halo acetate.

6. The process according to claim 3, wherein each of the transformations of alkylation and Dieckmann Condensation is carried out in the presence of at least one base.

7. The process according to claim 6, wherein the amount of base used in total is between 3.0 to 6.0 equivalents.

8. The process according to claim 2, wherein the compound of formula (5) is prepared by an asymmetric allylic alkylation from a compound of the following formula (3):

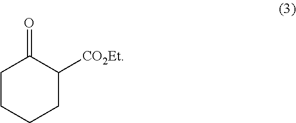 [SMILES: CCOC(=O)C1CCCCC1=O]

9. The process according to claim 8, where the asymmetric allylic alkylation of a compound of formula (3) is carried out in the presence of allylpalladium(II) chloride dimer below 0.250 mol % and (S,S)-DACH-Ph Trost ligand in a Pd/Ligand ratio of between 1.00:1.00 to 1.00:3.00.

10. The process according to claim 8, where the asymmetric allylic alkylation of (3) is carried out between 5 to 20° C. reaction temperature.

11. A process for the preparation of a compound of formula (10) or (11) 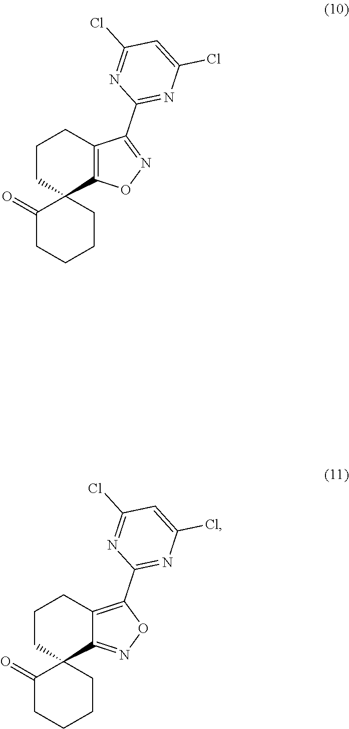 [SMILES: O=C1CCCC[C@@]12CCCc1c(-c3nc(Cl)cc(Cl)n3)noc12 ; O=C1CCCC[C@@]12CCCc1c2noc1-c1nc(Cl)cc(Cl)n1]
wherein the process comprises using the compound of formula (1) according to claim 1.

12. A process for the preparation of a compound of formula (10) 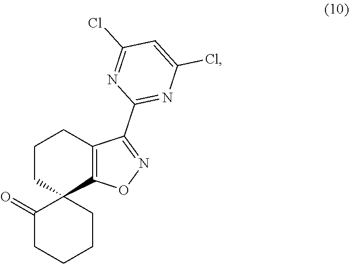 [SMILES: O=C1CCCC[C@@]12CCCc1c(-c3nc(Cl)cc(Cl)n3)noc12]
wherein the process comprises using the compound of the following formula (9) as intermediate:

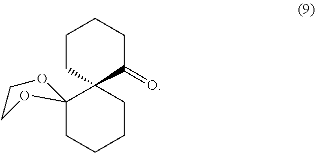 [SMILES: O=C1CCCC[C@@]12CCCCC21OCCO1]

13. The process according to claim 12 wherein compound of formula (9) is generated according to the process of claim 2, wherein the one-pot reaction of a compound of formula (5) has a compound of formula (9) as an isolated product and comprises 5 transformations: hydroboration, alkylation, Dieckmann Condensation, saponification and decaboxylation.

14. A compound of formula (10) or (11): 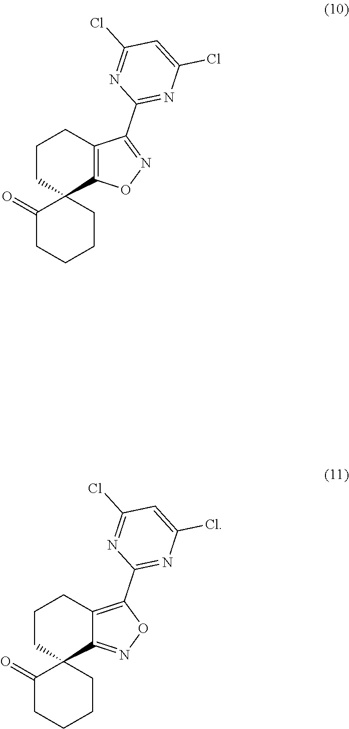 [SMILES: O=C1CCCC[C@@]12CCCc1c(-c3nc(Cl)cc(Cl)n3)noc12 ; O=C1CCCC[C@@]12CCCc1c2noc1-c1nc(Cl)cc(Cl)n1]
